(19) United States
(12) Patent Application Publication
(10) Pub. No.: US 2009/0054483 A1
(43) Pub. Date: Feb. 26, 2009

(54) Substituted Quinolines as Inhibitors of Leukotriene Biosynthesis

(76) Inventors: Yves Ducharme, Montreal (CA); Tom Yao-Hsiang Wu, Laval (CA); Richard Frenette, Laval (CA)

Correspondence Address: MERCK AND CO., INC, P O BOX 2000, RAHWAY, NJ, 07065-0907, US

(21) Appl. No.: 11/992,989

(22) PCT Filed: Oct. 2, 2006
(86) PCT No.: PCT/CA2006/001629

§ 371 Date: Apr. 2, 2008

Related U.S. Application Data
(60) Provisional application No. 60/723,780, filed on Oct. 5, 2005.

(51) Int. Cl. A61K 31/4709; A61P 29/00; A61P 9/10; A61P 11/06; C07D 413/12

(52) U.S. Cl. 514/314; 546/173

(57) ABSTRACT

The instant invention provides compounds of Formula (I) which are leukotriene biosynthesis inhibitors. Compounds of Formula (I) are useful as anti-atherosclerotic, anti-asthmatic, anti-allergic, anti-inflammatory and cytoprotective agents.

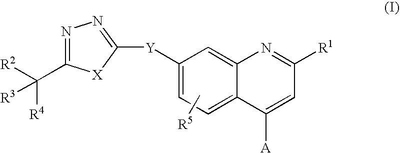

### FIELD OF THE INVENTION

The instant invention involves novel compounds which are useful as inhibitors of leukotriene biosynthesis.

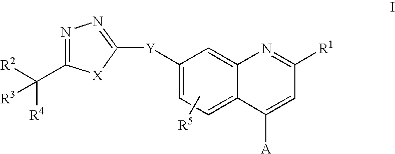

### BACKGROUND OF THE INVENTION

Inhibition of leukotriene biosynthesis has been an active area of pharmaceutical research for many years. The leukotrienes constitute a group of locally acting hormones, produced in living systems from arachidonic acid. Leukotrienes are potent contractile and inflammatory mediators derived by enzymatic oxygenation of arachidonic acid by 5-lipoxygenase. One class of leukotriene biosynthesis inhibitors are those known to act through inhibition of 5-lipoxygenase (5-LO).

The major leukotrienes are Leukotriene B₄ (abbreviated as LTB₄), LTC₄, LTD₄ and LTE₄. The biosynthesis of these leukotrienes begins with the action of the enzyme 5-lipoxygenase on arachidonic acid to produce the epoxide known as Leukotriene A₄ (LTA₄), which is converted into the other leukotrienes by subsequent enzymatic steps. Further details of the biosynthesis as well as the metabolism of the leukotrienes are to be found in Leukotrienes and Lipoxygenases, ed. J. Rokach, Elsevier, Amsterdam (1989). The actions of the leukotrienes in living systems and their contribution to various diseases states are also discussed in the Rokach text.

In general, 5-LO inhibitors have been sought for the treatment of allergic rhinitis, asthma, and inflammatory conditions, including arthritis. One example of a 5-LO inhibitor is the marketed drug zileuton (ZYLOFT®) which is indicated for the treatment of asthma. More recently, it has been reported that 5-LO may be an important contributor to the atherogenic process [see Mehrabian, M. et al., Circulation Research, 91:120-126 (2002)].

Despite significant therapeutic advances in the treatment and prevention of conditions affected by 5-LO inhibition, further treatment options are needed. The instant invention addresses that need by providing novel 5-LO inhibitors which are useful for inhibiting leukotriene biosynthesis.

### SUMMARY OF THE INVENTION

The instant invention relates to compounds of Formula I which are leukotriene biosynthesis inhibitors, methods for their preparation, and methods and pharmaceutical formulations for using these compounds in mammals, especially humans.

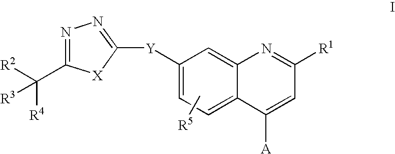

The compounds of Formula I are useful as pharmaceutical agents to slow or halt atherogenesis. Therefore, the instant invention provides a method for treating atherosclerosis, which includes halting or slowing the progression of atherosclerotic disease once it has become clinically evident, comprising administering a therapeutically effective amount of a compound of Formula I to a patient in need of such treatment. The instant invention also provides methods for preventing or reducing the risk of developing atherosclerosis and atherosclerotic disease events, comprising administering a prophylactically effective amount of a compound of Formula I to a patient who is at risk of developing atherosclerosis or having an atherosclerotic disease event.

Additionally, the instant invention involves the use of compounds of Formula I as anti-asthmatic, anti-allergic, anti-inflammatory and cytoprotective agents. They are also useful in treating angina, cerebral spasm, glomerular nephritis, hepatitis, endotoxemia, uveitis, and allograft rejection. The instant invention provides methods of treatment comprising administering a therapeutically effective amount of a compound of Formula I to a patient in need of the above-described treatments.

The instant invention further provides the use of a compound of Formula I in combination with other therapeutically effective agents. Additional embodiments will be evident from the following detailed description.

### DETAILED DESCRIPTION OF THE INVENTION

The novel leukotriene biosynthesis inhibitors of the instant invention are compounds of structural Formula I:

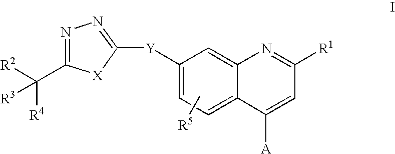

and pharmaceutically acceptable salts and solvates thereof, wherein:
n is 0, 1, or 2;
“A” is selected from the group consisting of
(a) a 5-membered aromatic ring containing (i) one or more carbon atoms, (ii) one heteroatom selected from oxygen and sulfur, and (iii) zero, one, two or three nitrogen atoms,
(b) a 5-membered aromatic ring containing one or more carbon atoms and from one to four nitrogen atoms,
(c) a 6-membered aromatic ring containing carbon atoms and one, two or three nitrogen atoms;
(d) a bicyclic aromatic ring system selected from benzothienyl, indolyl, quinolinyl and naphthalenyl;
(e) phenyl, and
(f) benzyl;
wherein A is optionally mono- or di-substituted with a substituent independently selected at each occurrence from the group consisting of (i) fluorine, (ii) chlorine, (iii) C₁₋₃ alkyl optionally substituted with one to five fluorines, (iv) C₁₋₃ alkoxy optionally substituted with one to five fluorines, (v) C₃₋₆ cycloalkyloxy, (vi) —CH₂OH, (vii) —COOR¹¹, (viii) cyano, (ix) hydroxy, and (x) —NR⁹R¹⁰;
Y is selected from:
(a) NR⁶—CHR⁷ wherein the nitrogen in Y is linked to the 5-membered heterocyclic moiety of Formula I and the carbon in Y is linked to the quinoline moiety of Formula I; and (b) S(O)ₙ;
X is selected from O and S;
each R¹¹ is independently selected from the group consisting of hydrogen, C₁₋₆ alkyl, and C₃₋₆ cycloalkyl;
R¹ is selected from the group consisting of cyano and —CONR¹¹R¹¹;
R² is selected from the group consisting of hydrogen, hydroxy, fluorine, C₁₋₃ alkyl, C₁₋₃ alkoxy, and C₁₋₃ alkylcarbonyloxy;
R³ is selected from the group consisting of hydrogen, C₁₋₆ alkyl optionally substituted with R⁸ or one to five fluorines, C₂₋₆ alkenyl, C₃₋₆ cycloalkyl, C₅₋₇ cycloalkenyl, and -Z;
R⁴ is selected from the group consisting of hydrogen, C₁₋₆ alkyl optionally substituted with R⁸ or one to five fluorines, C₂₋₆ alkenyl, C₃₋₆ cycloalkyl, C₅₋₇ cycloalkenyl, and -Z;
or R³ and R⁴ together represent oxo;
or R³ and R⁴ are joined together with the carbon to which they are attached to form a ring selected from C₃₋₆ cycloalkyl and C₅₋₇ cycloalkenyl, provided that when R³ and R⁴ are joined together with the carbon to which they are attached to form a C₅₋₇ cycloalkenyl ring, there is no double bond at the C-1 position in the ring;
or R², R³, and R⁴ are joined together with the carbon to which they are attached to form a cycloalkenyl ring selected from:

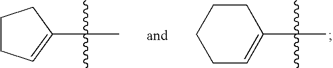

R⁵ is selected from the group consisting of hydrogen, C₁₋₆ alkyl, C₃₋₆ cycloalkyl, and halogen;
R⁶ is selected from the group consisting of hydrogen, C₁₋₄ alkyl, C₁₋₄ alkylcarbonyl, and benzoyl optionally substituted with C₁₋₄ alkyl;
R⁷ is selected from the group consisting of (a) hydrogen, (b) C₁₋₄ alkyl, (c) C₃₋₆ cycloalkyl, (d) phenyl optionally mono- or di-substituted with a substituent independently selected at each occurrence from the group consisting of C₁₋₄ alkyl and fluorine, and (e) a 5-membered aromatic ring containing (i) one or more carbon atoms, (ii) one heteroatom selected from oxygen and sulfur, and (iii) zero, one, two or three nitrogen atoms;
R⁸ is selected from the group consisting of —COOR¹¹, —C(O)H, cyano, —CR¹¹R¹¹OH, —OR¹¹, C₁₋₆ alkylthio, and C₃₋₆ cycloalkylthio;
R⁹ is selected from the group consisting of hydrogen, C₁₋₆ alkyl, C₃₋₆ cycloalkyl, and —COOR^{a};
R^{a} is C₁₋₆ alkyl or C₃₋₆ cycloalkyl;
R¹⁰ is selected from the group consisting of hydrogen, C₁₋₆ alkyl, and C₃₋₆ cycloalkyl; and
Z is selected from the group consisting of
(a) a 5-membered aromatic ring containing (i) one or more carbon atoms, (ii) one heteroatom selected from oxygen and sulfur, and (iii) zero, one, two or three nitrogen atoms,
(b) a 5-membered aromatic ring containing one or more carbon atoms and from one to four nitrogen atoms,
(c) a 6-membered aromatic ring containing carbon atoms and one, two or three nitrogen atoms;
(d) phenyl,
(e) benzyl, and (f) —CH₂-dioxolanyl;
and wherein Z is optionally mono- or di-substituted with a substituent independently selected at each occurrence from the group consisting of (i) fluorine, (ii) chlorine, (iii) C₁₋₃ alkyl optionally substituted with one to five fluorines, (iv) C₁₋₃ alkoxy optionally substituted with one to five fluorines, (v) C₃₋₆ cycloalkyloxy, (vi) —CH₂OH, (vii) —COOR¹¹, (viii) cyano, and (ix) —NR⁹R¹⁰.

In one embodiment of the present invention are compounds within the scope of Formula I, having the following structural Formula II:

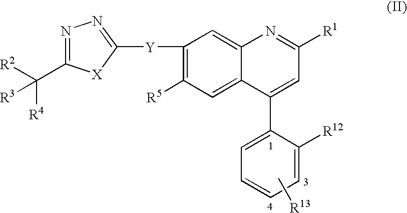

and pharmaceutically acceptable salts and solvates thereof, wherein:
R¹² is selected from the group consisting of hydrogen and fluorine; and
R¹³ is a substituent at the 3- or 4-position of the phenyl ring and is selected from the group consisting of (i) fluorine, (ii) C₁₋₃ alkyl optionally substituted with one to five fluorines, (iii) C₁₋₃ alkoxy optionally substituted with one to five fluorines, (iv) C₃₋₆ cycloalkyloxy, (v) —CH₂OH, (vi) —COOR¹¹, (vii) cyano, and (viii) —NR⁹R¹⁰; and the remaining variables are as defined in Formula I above. In a class of this embodiment, R¹³ is selected from the group consisting of hydrogen, fluorine, trifluoromethoxy, difluoromethoxy, cyano, methyl, and methoxy. In a subclass of this class, R¹³ is hydrogen or fluorine.

In another class of this embodiment are compounds within the scope of Formula II having the following structural Formula III:

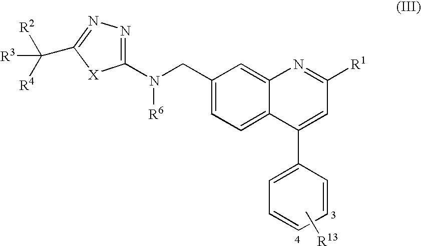

and pharmaceutically acceptable salts and solvates thereof wherein the variables present in Formula III are as defined in Formula II.

In another class of this embodiment are compounds within the scope of Formula II having the following structural Formula IV:

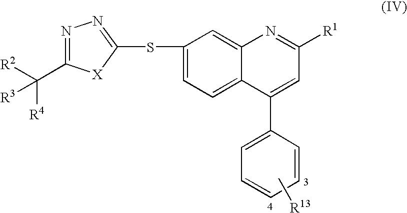

and pharmaceutically acceptable salts and solvates thereof wherein the variables present in Formula IV are as defined in Formula II.

In another embodiment of this invention are compounds of Formula I wherein A is selected from the group consisting of:

a) a 5-membered aromatic ring containing (i) one or more carbon atoms, (ii) one heteroatom selected from oxygen and sulfur, and (iii) zero, one, two or three nitrogen atoms,
b) a 5-membered aromatic ring containing one or more carbon atoms and from one to four nitrogen atoms,
c) a 6-membered aromatic ring containing carbon atoms and one, two or three nitrogen atoms, and
d) phenyl,
and wherein A is unsubstituted, mono- or di-substituted as described above for Formula I. In a class of this embodiment, A is unsubstituted, mono- or di-substituted as described above for Formula I, and is selected from the group consisting of thienyl, furanyl, oxazolyl, thiazolyl, tetrazolyl, pyridinyl and phenyl, and particularly thiazolyl, pyridinyl, and phenyl. In a class of this embodiment, A is phenyl, optionally substituted at the 3- or 4-position with a substituent independently selected from fluorine, trifluoromethoxy, difluoromethoxy, cyano, methyl, and methoxy, and optionally substituted at the 2-position with fluorine. More particularly, A is 4-fluorophenyl.

In another embodiment of this invention are compounds of Formulae I, II, III, and IV wherein X is S. In another embodiment are compounds of Formulae I, II, III, and IV wherein X is O.

In another embodiment of this invention are compounds of Formula I wherein Y is NR⁶—CHR⁷. In a class of this embodiment, Y is NR⁶—CH₂. In another embodiment are compounds of Formula I wherein Y is S.

In another embodiment of this invention are compounds of Formula I wherein R¹¹ is hydrogen or C₁₋₆ alkyl. In a class of this embodiment, R¹¹ is hydrogen or methyl, and in a subclass, R¹¹ is hydrogen.

In another embodiment of this invention are compounds of Formulae I, II, III, and IV wherein R² is selected from the group consisting of hydrogen, hydroxy, fluorine, C₁₋₃ alkyl, methoxy, and methylcarbonyloxy. In a class of this embodiment, R² is hydrogen or hydroxy, and more particularly it is hydroxy.

In another embodiment of this invention are compounds of Formulae I, II, III, and IV wherein R³ is selected from the group consisting of hydrogen, C₁₋₆ alkyl optionally substituted with one to five fluorines, C₃₋₆ cycloalkyl, and phenyl. In a class of this embodiment, R³ is cyclopropyl or C₁₋₂ alkyl wherein alkyl is optionally substituted with one to five fluorines.

In another embodiment of this invention are compounds of Formulae I, II, III, and IV wherein R⁴ is selected from the group consisting of hydrogen, C₃₋₆ cycloalkyl, and C₁₋₆ alkyl optionally substituted with R⁸ or one to five fluorines. In a class of this embodiment, R⁴ is selected from C₁₋₂ alkyl optionally substituted with one to five fluorines, cyclopropyl, and —CH₂COOC₁₋₄ alkyl.

In another embodiment of this invention are compounds of Formulae I, II, III, and IV wherein R³ and R⁴ together represent oxo, or R³ and R⁴ together with the carbon to which they are attached represent a C₃₋₆ cycloalkyl ring.

In another embodiment of this invention are compounds of Formulae I, II, III and IV wherein R² is hydroxy, R³ is ethyl and R⁴ is —CF₃, forming a 1-hydroxy-1-(trifluoromethyl)propyl-group, and particularly the (S)-stereoisomer of 1-hydroxy-1-(trifluoromethyl)propyl-, shown below attached to the oxadiazolyl or thiadiazolyl ring in the structure:

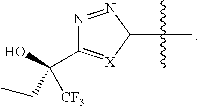

In another embodiment of this invention are compounds of Formulae I and II wherein R⁵ is selected from hydrogen, methyl, fluorine, and chlorine. In a class of this embodiment, R⁵ is hydrogen or methyl.

In another embodiment of this invention are compounds of Formulae I, II, and III wherein R⁶ is selected from the group consisting of hydrogen, methyl, and methylcarbonyl. In a class of this embodiment, R⁶ is hydrogen.

In another embodiment of this invention are compounds of Formulae I, II, and III wherein R⁷ is selected from the group consisting of hydrogen and C₁₋₄ alkyl. In a class of this embodiment, R⁷ is hydrogen.

In another embodiment of this invention are compounds of Formulae I, II, III, and IV wherein R⁸ is —COOR¹¹, and particularly —COOC₁₋₆ alkyl.

In another embodiment of this invention are compounds of Formulae I, II, III, and IV wherein R⁹ is selected from hydrogen and C₁₋₆ alkyl.

In another embodiment of this invention are compounds of Formulae I, II, III, and IV wherein R¹⁰ is selected from hydrogen and C₁₋₆ alkyl.

In another embodiment of this invention are compounds of Formulae I, II, III, and IV wherein Z is unsubstituted, mono- or di-substituted as described in Formula I and is selected from the group consisting of phenyl, benzyl, pyridinyl, thiazolyl, dioxolanyl, and tetrazolyl. In a class of this embodiment, Z is unsubstituted, mono- or di-substituted and is selected from the group consisting of phenyl, pyridinyl and thiazolyl.

Illustrative, but non-limiting, examples of compounds of the present invention that are useful as inhibitors of leukotriene biosynthesis are the following:

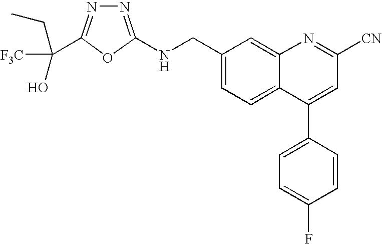

4-(4-fluorophenyl)-7-[({5-[1-hydroxy-1-(trifluoromethyl)propyl]-1,3,4-oxadiazol-2-yl}amino)methyl]quinoline-2-carbonitrile;

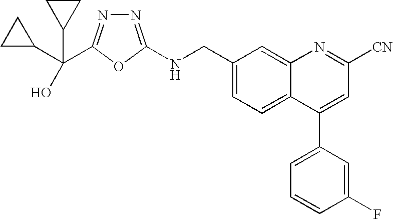

7-[({5-[dicyclopropyl(hydroxy)methyl]-1,3,4-oxadiazol-2-yl}amino)methyl]-4-(3-fluorophenyl)quinoline-2-carbonitrile;

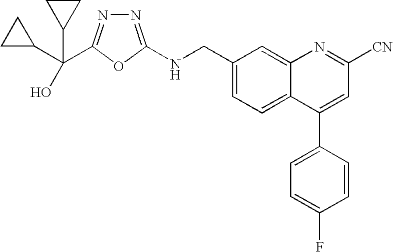

7-[({5-[dicyclopropyl(hydroxy)methyl]-1,3,4-oxadiazol-2-yl}amino)methyl]-4-(4-fluorophenyl)quinoline-2-carbonitrile;

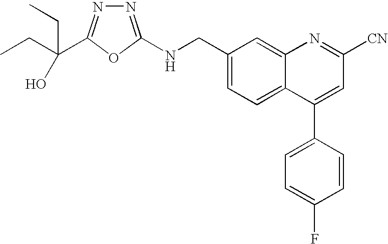

7-({[5-(1-ethyl-1-hydroxypropyl)-1,3,4-oxadiazol-2-yl]amino}methyl)-4-(4-fluorophenyl)quinoline-2-carbonitrile;

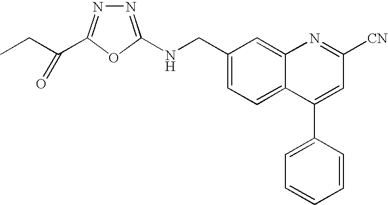

4-phenyl-7-{[(5-propionyl-1,3,4-oxadiazol-2-yl)amino]methyl}quinoline-2-carbonitrile;

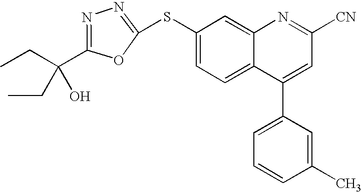

7-{[5-(1-ethyl-1-hydroxypropyl)-1,3,4-oxadiazol-2-yl]thio}-4-(3-methylphenyl)quinoline-2-carbonitrile;

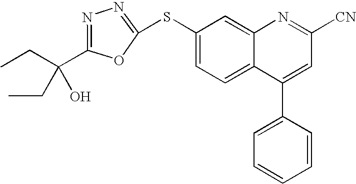

7-{[5-(1-ethyl-1-hydroxypropyl)-1,3,4-oxadiazol-2-yl]thio}-4-phenylquinoline-2-carbonitrile;

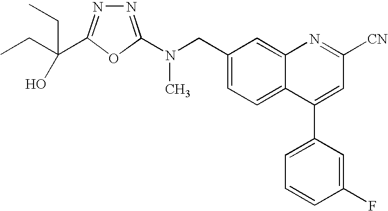

7-{[[5-(1-ethyl-1-hydroxypropyl)-1,3,4-oxadiazol-2-yl](methyl)amino]methyl}-4-(3-fluorophenyl)quinoline-2-carbonitrile;

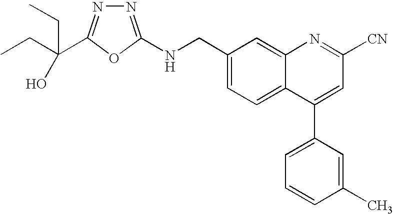

7-({[5-(1-ethyl-1-hydroxypropyl)-1,3,4-oxadiazol-2-yl]amino}methyl)-4-(3-methylphenyl)quinoline-2-carbonitrile;

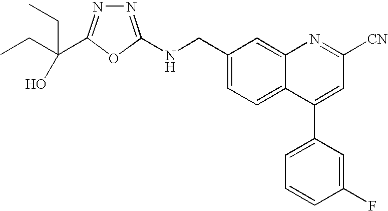

7-({[5-(1-ethyl-1-hydroxypropyl)-1,3,4-oxadiazol-2-yl]amino}methyl)-4-(3-fluorophenyl)quinoline-2-carbonitrile;

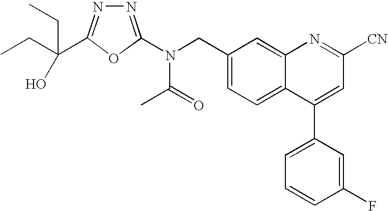

N-{[2-cyano-4-(3-fluorophenyl)quinolin-7-yl]methyl}-N-[5-(1-ethyl-1-hydroxypropyl)-1,3,4-oxadiazol-2-yl]acetamide;

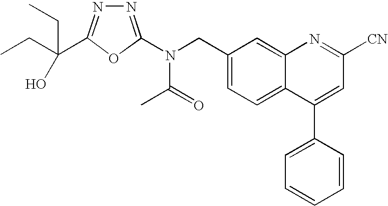

N-[(2-cyano-4-phenylquinolin-7-yl)methyl]-N-[5-(1-ethyl-1-hydroxypropyl)-1,3,4-oxadiazol-2-yl]acetamide;

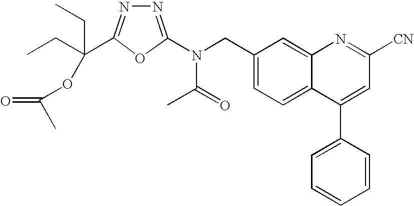

1-(5-{acetyl[(2-cyano-4-phenylquinolin-7-yl)methyl]amino}-1,3,4-oxadiazol-2-yl)-1-ethylpropyl acetate;

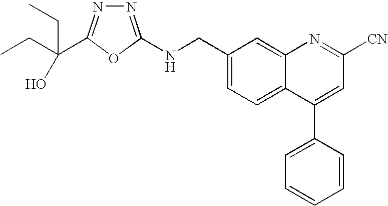

7-({[5-(1-ethyl-1-hydroxypropyl)-1,3,4-oxadiazol-2-yl]amino}methyl)-4-phenylquinoline-2-carbonitrile;

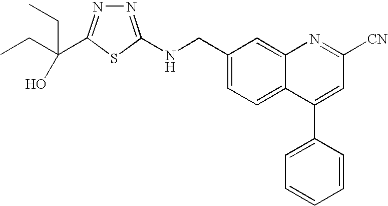

7-({[5-(1-ethyl-1-hydroxypropyl)-1,3,4-thiadiazol-2-yl]amino}methyl)-4-phenylquinoline-2-carbonitrile;
   and pharmaceutically acceptable salts and solvates thereof.

The compounds of this invention, including compounds referenced as those of “Formula I,” “Formula II,” “Formula III,” “Formula IV,” or any other generic structural formulas used herein to describe the compounds of this invention, are intended to encompass compounds falling within the scope of each of these structural formulas including pharmaceutically acceptable salts and solvates thereof where such salts and solvates are possible.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl can be employed. Included are those esters and acyl groups known in the art for modifying the solubility or hydrolysis characteristics for use as sustained-release or prodrug formulations.

Some of the compounds described herein contain one or more asymmetric centers and may thus give rise to diastereomers and optical isomers. The present invention is meant to comprehend such possible diastereomers as well as their racemic and resolved, enantiomerically pure forms and pharmaceutically acceptable salts thereof. Furthermore, some of the crystalline forms of compounds of the present invention may exist as polymorphs and as such are intended to be included in the present invention. In addition, some of the compounds of the instant invention may form solvates with water or common organic solvents. Such solvates and hydrates are likewise encompassed within the scope of this invention. Some of the compounds described herein contain olefinic double bonds. The invention includes both E and Z geometric isomers.

Compounds of this invention may be separated into their individual diastereoisomers by, e.g., fractional crystallization from suitable solvents, e.g., methylene chloride/hexanes or EtOAc/hexanes, or via chiral chromatography using an optically active stationary phase. Absolute stereochemistry may be determined by X-ray crystallography of crystalline products or crystalline intermediates which are derivatized, if necessary, with a reagent containing a stereogenic center of known configuration. Alternatively, any stereoisomer of a compound of this invention may be obtained by stereospecific synthesis using optically pure starting materials or reagents of known absolute configuration.

As used herein “alkyl” is intended to include both branched- and straight-chain saturated aliphatic hydrocarbon groups having the specified number of carbon atoms, e.g., methyl (Me), ethyl (Et), n-propyl (Pr), n-butyl (Bu), n-pentyl, n-hexyl, and the isomers thereof such as isopropyl (i-Pr), isobutyl (i-Bu), secbutyl (s-Bu), tertbutyl (t-Bu), isopentyl, isohexyl and the like. “Cycloalkyl” means a monocyclic saturated carbocyclic ring, having the specified number of carbon atoms, e.g., 3, 4, 5 or 6 carbon atoms. Examples of cycloalkyl include cyclopropyl, cyclobutyl, cyclopentyl and cyclohexyl.

The term “C₂₋₆ alkenyl” as used herein, refers to a straight or branched 2-6 carbon chain with at least one carbon-carbon double bond. Examples of alkenyl include, but are not limited to, vinyl (—CH═CH₂), allyl, isopropenyl, pentenyl, hexenyl, heptenyl, 1-propenyl, 2-butenyl, 2-methyl-2-butenyl, and the like. The term “C₅₋₇ cycloalkenyl” as used herein means a non-aromatic monocyclic ring having from 5 to 7 carbon atoms in the ring with at least one carbon-carbon double bond.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

The term “alkylcarbonyloxy” refers to straight or branched chain alkylacyl derivatives of alcohol (OH) groups found in the compounds the present invention of the number of carbon atoms specified (e.g., C₁₋₆ alkylcarbonyloxy), or any number within this range [i.e., methylcarbonyloxy (MeCOO—), ethylcarbonyloxy, or butylcarbonyloxy].

Within the definition of variables above, R³ and R⁴ can be joined together with the carbon to which they are attached to form a C₅₋₇ cycloalkenyl ring wherein there is no double bond at the C-1 position in the ring. The C-1 position is intended to be the ring carbon in the cycloalkenyl ring that is bonded to the core oxadiazolyl or thiadiazolyl ring in the generic structural formulas depicted herein. In this situation, C-1 is also bonded to R². This is illustrated below using the example where R³ and R⁴ are joined together with the carbon to which they are attached to form a 3,4-cyclopentenyl ring, see (a):

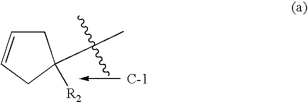

The term “optionally substituted” means “unsubstituted or substituted,” and therefore, the generic structural formulas described herein encompasses compounds containing the specified optional substituents as well as compounds that do not contain the optional substituents. For example, the phrase “benzoyl optionally substituted with C₁₋₄ alkyl” encompasses unsubstituted benzoyl and benzoyl substituted with C₁₋₄ alkyl. Each variable is independently defined each time it occurs within the generic structural formula definitions. For example, when R⁸ is —CR¹¹R¹¹OH, R¹¹ is independently selected at each occurrence and each R¹¹ can be the same or different.

Use of the term “substituted” is intended to encompass mono- and poly-substitution on the specified moiety, unless otherwise specified. A mono-substituted moiety has one substituent, while a polysubstituted moiety has more than one substituent wherein each carbon atom, as well as heteroatom such as nitrogen if present, that is available for substitution in the moiety may independently be unsubstituted, mono- or polysubstituted and which results in the creation of a stable structure. For example, “C₁₋₆ alkyl optionally substituted with fluorine” includes CH₃, CH₂F, CHF₂ and CF₃.

The terms “halo” or “halogen” are meant to include fluorine, chlorine, bromine and iodine, unless otherwise noted. Fluorine and chlorine are preferred, and fluorine is most preferred.

Examples of 5-membered aromatic rings within the definitions of A and Z include but are not limited to thienyl, furanyl, oxazolyl, thiazolyl, pyrrolyl, pyrazolyl, imidazolyl, and tetrazolyl, represented by the structural formulas below:

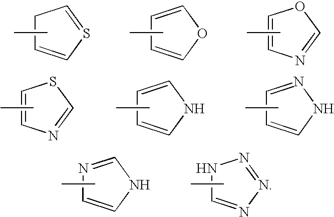

Examples of 6-membered aromatic rings comprised of carbon and one, two or three nitrogens within the definition of A and Z include but are not limited to pyridinyl, pyrimidinyl, pyrazinyl and triazinyl represented by the structural formulas below:

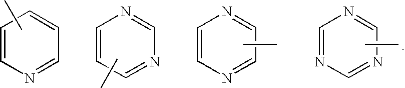

The ability of the compounds of this invention to inhibit biosynthesis of the leukotrienes makes them useful for preventing or reversing the symptoms induced by the leukotrienes in a human subject. Accordingly, this invention provides a method for preventing the synthesis, the action, or the release of leukotrienes in a mammal which comprises administering to said mammal a 5-LO inhibitory effective amount of a compound of this invention. Such 5-LO inhibitory activity can be measured using the Human 5-Lipoxygenase Enzyme Assay and 5-Lipoxygenase Human Whole Blood Assay described herein. Since leukotrienes are potent inflammatory mediators, also provided is method of treating an inflammatory condition in a mammal which comprises administering a therapeutically effective amount of a compound of this invention to a mammal in need of such treatment.

The inhibition of the mammalian biosynthesis of leukotrienes also indicates that the compounds and pharmaceutical compositions thereof are useful to treat, prevent or ameliorate atherosclerosis in mammals, and especially in humans. Therefore, the compounds of this invention can be used for the treatment of atherosclerosis comprising administering a therapeutically effective amount of a compound of this invention to a patient in need of such treatment.

The method of this invention serves to prevent or slow new atherosclerotic lesion or plaque formation, and to prevent or slow progression of existing lesions or plaques, as well as to cause regression of existing lesions or plaques. Accordingly, one aspect of this invention encompassed within the scope of treatment of atherosclerosis involves a method for halting or slowing the progression of atherosclerosis, including halting or slowing atherosclerotic plaque progression, comprising administering a therapeutically effective amount of a compound of this invention to a patient in need of such treatment. This method includes halting or slowing progression of atherosclerotic plaques existing at the time the instant treatment is begun (i.e., “existing atherosclerotic plaques”), as well as halting or slowing formation of new atherosclerotic plaques in patients with atherosclerosis.

Another aspect of this invention encompassed within the scope of treatment of atherosclerosis involves a method for effecting regression of atherosclerosis, including effecting regression of atherosclerotic plaques existing at the time the instant treatment is begun, comprising administering a therapeutically effective amount of a compound of this invention to a patient in need of such treatment.

Also provided is a method comprising administering to a patient who has atherosclerosis a compound of this invention with the objective of preventing or reducing the risk of atherosclerotic plaque rupture. Therefore, this invention provides a method for preventing or reducing the risk of atherosclerotic plaque rupture comprising administering a prophylactically effective amount of a compound of this invention to a patient having atherosclerotic plaque.

This invention also involves a method for preventing or reducing the risk of developing atherosclerosis, comprising administering a prophylactically effective amount of a compound of this invention to a patient in need of such treatment, including. for example, a patient who is at risk for developing atherosclerosis.

Atherosclerosis is characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction, are all clinical manifestations of atherosclerosis and are therefore encompassed by the terms “atherosclerosis” and “atherosclerotic disease.”

A compound of the instant invention may be administered to prevent or reduce the risk of occurrence, or recurrence where the potential exists, of a coronary heart disease (CHD) event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of this invention to a patient in need of such treatment, such as a patient who is at risk for such an event. The patient in need of such treatment may already have atherosclerotic disease at the time of administration, or may be at risk for developing it.

This invention also provides a method for treating, preventing, or ameliorating angina and/or myocardial ischemia, comprising administering a therapeutically or prophylactically effective amount, as appropriate, of a compound of this invention to a patient in need of such treatment.

Additionally, the activity of the instant compounds as leukotriene biosynthesis inhibitors makes them useful for treating, preventing, or ameliorating: 1) pulmonary disorders including diseases such as asthma, chronic bronchitis, and related obstructive airway diseases, 2) allergies and allergic reactions such as allergic rhinitis, contact dermatitis, allergic conjunctivitis, and the like, 3) inflammation such as arthritis or inflammatory bowel disease, 4) pain, 5) skin disorders such as atopic eczema, and the like, 6) cardiovascular disorders such hypertension, platelet aggregation and the like, 7) renal insufficiency arising from ischaemia induced by immunological or chemical (cyclosporin) etiology and 8) migraine or cluster headache, 9) ocular conditions such as uveitis, 10) hepatitis resulting from chemical, immunological or infectious stimuli, 11) trauma or shock states such as burn injuries, endotoxemia and the like, 12) allograft rejection, 13) prevention of side effects associated with therapeutic administration of cytokines such as Interleukin II and tumor necrosis factor, 14) chronic lung diseases such as cystic fibrosis, bronchitis and other small- and large-airway diseases, 15) cholecystitis, 16) multiple sclerosis, 17) proliferation of myoblastic leukemia cells, 18) pulmonary fibrosis, 19) respiratory syncytial virus, 20) acne and 21) sleep apnea. Moreover, the compounds of this invention can be administered to patients, including adult and pediatric patients, for the relief of symptoms of allergic rhinitis, including seasonal allergic rhinitis.

Particularly, the compounds of this invention can be administered to patients, including adult and pediatric patients, for the prophylaxis of asthma and for chronic treatment of asthma.

The compounds of this invention can be administered to patients, including adult and pediatric patients, for the treatment of asthma: (1) as an alternative to low-dose inhaled corticosteroids (ICS) for patients with mild persistent asthma, (2) as concomitant therapy with low-dose inhaled corticosteroids (ICS) for patients with mild persistent asthma, or (3) as concomitant therapy in patients with persistent asthma who are inadequately controlled on inhaled corticosteroids (ICS) or on combined ICS/long-acting beta-agonist (LABA) therapy. The compounds can be used for treatment of asthmatic patients including, but not limited to, steroid resistant/non-responder asthmatics, asthmatics for whom leukotriene modifiers have previously failed, smoking asthmatics, and aspirin sensitive asthmatics.

The compounds can be administered to patients to: (1) improve FEVL (Forced Expitory Volume in one minute), (2) improve morning and evening PEF (Peak Expitory flow), (3) reduce beta-agonist use (measured by puffs/day), (4) reduce inhaled/systemic steroid use. (5) improve daytime asthma symptoms, (6) reduce number of nocturnal awakenings, 7) improve asthma control days, (8) reduce number of asthma exacerbations, wherein an exacerbation is defined as: requiring systemic steroid, an emergency room visit, hospitalization, an unscheduled asthma related doctor visit, decrease in A.M. PEF by >20% or A.M. PEF <180 l/min, increased SABA (short-acting beta-agonist) use >70% from baseline (minimum increase 2 puffs), or increased symptom score of >50%, (9) reduce the number of asthma attacks (measured as % of days with at least one attack over a specified period of total days), wherein the attack is one that requires systemic steroid use, an emergency room visit, hospitalization, or an unscheduled asthma related doctor visit, (10) reduce the number of acute asthma attacks, (11) reduce blood and sputum eosinophils, and/or (12) prevent and treat EIB (exercised induced bronchoconstriction).

The compounds of the present invention may also be used to treat or prevent mammalian (especially, human) disease states such as erosive gastritis; erosive esophagitis; diarrhea; cerebral spasm; premature labor; spontaneous abortion; dysmenorrhea; ischemia; noxious agent-induced damage or necrosis of hepatic, pancreatic, renal, or myocardial tissue; liver parenchymal damage caused by hepatoxic agents such as CCl₄ and D-galactosamine; ischemic renal failure; disease-induced hepatic damage; bile salt induced pancreatic or gastric damage; trauma- or stress-induced cell damage; and glycerol-induced renal failure. Leukotriene biosynthesis inhibitors also act as inhibitors of tumor metastasis and exhibit cytoprotective action.

The cytoprotective activity of a compound may be observed in both animals and man by noting the increased resistance of the gastrointestinal mucosa to the noxious effects of strong irritants, for example, the ulcerogenic effects of aspirin or indomethacin. In addition to lessening the effect of non-steroidal anti-inflammatory drugs on the gastrointestinal tract, animal studies show that cytoprotective compounds will prevent gastric lesions induced by oral administration of strong acids, strong bases, ethanol, hypertonic saline solutions, and the like. Two assays can be used to measure cytoprotective ability. These assays are: (A) an ethanol-induced lesion assay and (B) an indomethacin-induced ulcer assay and are described in EP 140,684. In particular, the compounds of the invention would be useful to reduce the gastric erosion caused by co-administration of a cyclooxygenase-2 selective inhibitor such as rofecoxib (VIOXX®), etoricoxib (ARCOXIA™), and celecoxib (CELEBREX®) and low-dose aspirin.

In addition, the compounds of this invention can also be used for the treatment of chronic obstructive pulmonary disease (COPD). As described in S. Kilfeather, Chest, 2002, vol 121, 197, airway neutrophilia in COPD patients is believed to be a contributing source of inflammation and is associated with airway remodeling. The presence of neutrophils is mediated in part by LTB₄, and treatment with the instant compounds could be used to reduce neutrophilic inflammation in patients with COPD and reduce the rate of COPD exacerbations. In particular, the compounds of this invention could be used for daily, preferably once-daily, maintenance treatment of airflow obstruction associated with COPD, including chronic bronchitis and emphysema.

Additionally, the 5-LO inhibitor compounds of this invention may be useful for treatment of psychiatric disorders, such as depression and anxiety; see Manev, R. and Manev, H., “5-Lipoxygenase as a Putative Link Between Cardiovascular and Psychiatric Disorders,” Critical Reviews in Neurobiology, 16: 181-186 (2004).

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by inhibition of leukotriene biosynthesis.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

The magnitude of prophylactic or therapeutic dose of a compound of this invention will, of course, vary with the nature of the severity of the condition to be treated and with the particular compound and its route of administration. It will also vary according to the age, weight and response of the individual patient. It is understood that a specific daily dosage amount can simultaneously be both a therapeutically effective amount, e.g., for treatment to slow progression of existing atherosclerosis, and a prophylactically effective amount, e.g., for prevention of an atherosclerotic disease event or formation of new lesions. In general, the daily dose range for anti-asthmatic, anti-inflammatory, anti-allergic or anti-atherosclerotic use and generally, uses other than cytoprotection, lie within the range of from about 0.001 mg to about 100 mg per kg body weight of a mammal, preferably 0.01 mg to about 10 mg per kg, and most preferably 0.1 to 1 mg per kg, in single or divided doses. On the other hand, it may be necessary to use dosages outside these limits in some cases.

In the case where an oral composition is employed, a suitable daily dosage range for anti-asthmatic, anti-inflammatory, anti-allergic or anti-atherosclerotic use is, e.g., from about 0.01 mg to about 100 mg of a compound of this invention per kg of body weight per day, and preferably from about 0.1 mg to about 10 mg per kg. For cytoprotective use a suitable daily dosage range is from 0.1 mg to about 100 mg, preferably from about 1 mg to about 100 mg, and more preferably from about 10 mg to about 100 mg, of a compound of this invention per kg of body weight per day.

For use where a composition for intravenous administration is employed, a suitable daily dosage range for anti-asthmatic, anti-inflammatory, anti-atherosclerotic or anti-allergic use is from about 0.001 mg to about 25 mg (preferably from 0.01 mg to about 1 mg) of a compound of this invention per kg of body weight per day and for cytoprotective use from about 0.1 mg to about 100 mg (preferably from about 1 mg to about 100 mg and more preferably from about 1 mg to about 10 mg) of a compound of this invention per kg of body weight per day. For the treatment of diseases of the eye, ophthalmic preparations for ocular administration comprising 0.001-1% by weight solutions or suspensions of the compounds of this invention in an acceptable ophthalmic formulation may be used.

The exact amount of a compound of this invention to be used as a cytoprotective agent will depend on, inter alia, whether it is being administered to heal damaged cells or to avoid future damage, on the nature of the damaged cells (e.g., gastrointestinal ulcerations vs. nephrotic necrosis), and on the nature of the causative agent. An example of the use of a compound of this invention in avoiding future damage would be co-administration of a compound of this invention with an NSAID that might otherwise cause such damage (for example, indomethacin). For such use, the compound of this invention is administered from 30 minutes prior up to 30 minutes after administration of the NSAID. Preferably it is administered prior to or simultaneously with the NSAID, (for example, in a combination dosage form).

The pharmaceutical compositions of the present invention comprise a compound of this invention as an active ingredient and a pharmaceutically acceptable carrier and optionally other therapeutic ingredients. Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. For use in treating or preventing atherosclerosis and related disease events, oral formulation is preferred.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery system for inhalation is a metered dose inhalation (MDI) aerosol, which may be formulated as a suspension or solution of a compound of this invention in suitable propellants, such as fluorocarbons or hydrocarbons.

Suitable topical formulations of a compound of this invention include transdermal devices, aerosols, creams, ointments, lotions, dusting powders, and the like.

In practical use, the compounds of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of this invention may also be administered by controlled release means and/or delivery devices such as those described in U.S. Pat. Nos. 3,845,770; 3,916,899; 3,536,809; 3,598,123; 3,630,200; 4,008,719; and 5,366,738 the disclosures of which are incorporated herein by reference.

Pharmaceutical compositions of the present invention suitable for oral administration may be presented as discrete units such as capsules, cachets or tablets, each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet, cachet or capsule contains from about 1 mg to about 500 mg of the active ingredient, for example but not limited to 10 mg, 20 mg, 30 mg, 40 mg, 50 mg and 75 mg. The following are examples of representative pharmaceutical dosage forms for the compounds of this invention:

| Injectable Suspension (I.M.) | mg/ml |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |
| Water for injection to a total volume of 1 ml | |

| Tablet | mg/tablet |
|---|---|
| Compound of Formula I | 25 |
| Microcrystalline Cellulose | 415 |
| Providone | 14.0 |
| Pregelatinized Starch | 43.5 |
| Magnesium Stearate | 2.5 |
| | 500 |

| Capsule | mg/capsule |
|---|---|
| Compound of Formula I | 25 |
| Lactose Powder | 573.5 |
| Magnesium Stearate | 1.5 |
| | 600 |

| Aerosol | Per canister | |
|---|---|---|
| Compound of Formula I | 24 | mg |
| Lecithin, NF Liquid Concentrate | 1-2 | mg |
| Trichlorofluoromethane, NF | 4.025 | gm |
| Dichlorodifluoromethane, NF | 12.15 | gm |

The instant invention also encompasses a process for preparing a pharmaceutical composition comprising combining a compound of this invention with a pharmaceutically acceptable carrier. Also encompassed is the pharmaceutical composition which is made by combining a compound of this invention with a pharmaceutically acceptable carrier.

A therapeutically effective amount of a compound of this invention can be used for the preparation of a medicament useful for treating or preventing any of the medical conditions described herein, in dosage amounts described herein. For example, a compound of this invention can be used for the preparation of a medicament useful for preventing or reducing the risk of developing atherosclerotic disease, halting or slowing the progression of atherosclerotic disease once it has become clinically manifest, and preventing or reducing the risk of a first or subsequent occurrence of an atherosclerotic disease event. Additionally, a compound of this invention can be used for the preparation of a medicament useful for the treatment of asthma, allergies and allergic conditions, inflammation, COPD or erosive gastritis. The medicament comprised of a compound of this invention may also be prepared with one or more additional active agents, such as those described below.

One or more additional active agents may be used in combination with the compounds of this invention in a single dosage formulation, or the active agents of the combination may be administered to the patient in separate dosage formulations, which allows for concurrent or sequential administration of the active agents. Unless otherwise specified, reference herein to compounds of this invention being used in combination with other active agents or used as part of combination therapy or the like encompasses both a single pharmaceutical composition comprised of a compound of this invention with one or more additional active agents, as well as a pharmaceutical composition comprised of a compound of this invention administered as part of a combination therapy with one or more other separately formulated active agents.

In addition to the compounds of this invention, the pharmaceutical compositions of the present invention can also contain other active agents (i.e., ingredients) and the pharmaceutical compositions comprised of a compound of this invention may be used for combination therapy with one or more other separately formulated active agents, such as cyclooxygenase inhibitors, non-steroidal anti-inflammatory drugs (NSAIDs), peripheral analgesic agents such as zomepirac diflunisal and the like. The weight ratio of the compound of this invention to the second active ingredient may be varied and will depend upon the effective dose of each ingredient. Generally, an effective dose of each will be used. Thus, for example, when a compound of this invention is combined with an NSAID the weight ratio of the compound of said compound to the NSAID will generally range from about 1000:1 to about 1:1000, preferably about 200:1 to about 1:200. Combinations of a compound of this invention and other active ingredients will generally also be within the aforementioned range, but in each case, an effective dose of each active ingredient should be used.

NSAIDs can be characterized into five groups: (1) propionic acid derivatives; (2) acetic acid derivatives; (3) fenamic acid derivatives; (4)oxicams; and (5)biphenylcarboxylic acid derivatives; or a pharmaceutically acceptable salt thereof.

The propionic acid derivatives which may be used comprise: alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen. Structurally related propionic acid derivatives having similar analgesic and anti-inflammatory properties are also intended to be included in this group. Thus, “propionic acid derivatives” as defined herein are non-narcotic analgesics/non-steroidal anti-inflammatory drugs having a free —CH(CH₃)COOH or —CH₂CH₂COOH group (which optionally can be in the form of a pharmaceutically acceptable salt group, e.g., —CH(CH₃)COO⁻Na⁺ or —CH₂CH₂COO⁻Na⁺), typically attached directly or via a carbonyl function to a ring system, preferably to an aromatic ring system.

The acetic acid derivatives which may be used comprise: indomethacin, which is a preferred NSAID, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac. Structurally related acetic acid derivatives having similar analgesic and anti-inflammatory properties are also intended to be encompassed by this group. Thus, “acetic acid derivatives” as defined herein are non-narcotic analgesics/non-steroidal anti-inflammatory drugs having a free —CH₂COOH group (which optionally can be in the form of a pharmaceutically acceptable salt group, e.g., —CH₂COO⁻Na⁺), typically attached directly to a ring system, preferably to an aromatic or heteroaromatic ring system.

The fenamic acid derivatives which may be used comprise: flufenamic acid, meclofenamic acid, mefenamic acid, niflumic acid and tolfenamic acid. Structurally related fenamic acid derivatives having similar analgesic and anti-inflammatory properties are also intended to be encompassed by this group. Thus, “fenamic acid derivatives” as defined herein are non-narcotic analgesics/non-steroidal anti-inflammatory drugs which contain the basic structure:

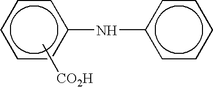

which can bear a variety of substituents and in which the free —COOH group can be in the form of a pharmaceutically acceptable salt group, e.g., —COO⁻Na⁺.

The biphenylcarboxylic acid derivatives which can be used comprise: diflunisal and flufenisal. Structurally related biphenyl-carboxylic acid derivatives having similar analgesic and anti-inflammatory properties are also intended to be encompassed by this group. Thus, “biphenylcarboxylic acid derivatives” as defined herein are non-narcotic analgesics/non-steroidal anti-inflammatory drugs which contain the basic structure:

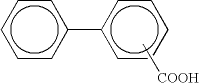

which can bear a variety of substituents and in which the free —COOH group can be in the form of a pharmaceutically acceptable salt group, e.g., —COO⁻Na⁺.

The oxicams which can be used in the present invention comprise: isoxicam, piroxicam, sudoxicam and tenoxican. Structurally related oxicams having similar analgesic and anti-inflammatory properties are also intended to be encompassed by this group. Thus, “oxicams” as defined herein are non-narcotic analgesics/non-steroidal anti-inflammatory drugs which have the general formula:

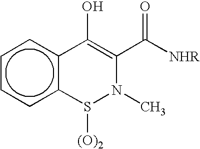

wherein R is an aryl or heteroaryl ring system.

The following NSAIDs may also be used: amfenac sodium, aminoprofen, anitrazafen, antrafenine, auranofin, bendazac lysinate, benzydanine, beprozin, broperamole, bufezolac, cinmetacin, ciproquazone, cloximate, dazidamine, deboxamet, delmetacin, detomidine, dexindoprofen, diacerein, di-fisalamine, difenpyramide, emorfazone, enfenamic acid, enolicam, epirizole, etersalate, etodolac, etofenamate, fanetizole mesylate, fenclorac, fendosal, fenflumizole, feprazone, floctafenine, flunixin, flunoxaprofen, fluproquazone, fopirtoline, fosfosal, furcloprofen, glucametacin, guaimesal, ibuproxam, isofezolac, isonixim, isoprofen, isoxicam, lefetamine HCl, leflunomide, lofemizole, lonazolac calcium, lotifazole, loxoprofen, lysin clonixinate, meclofenamate sodium, meseclazone, nabumetone, nictindole, nimesulide, orpanoxin, oxametacin, oxapadol, perisoxal citrate, pimeprofen, pimetacin, piproxen, pirazolac, pirfenidone, proglumetacin maleate, proquazone, pyridoxiprofen, sudoxicam, talmetacin, talniflumate, tenoxicam, thiazolinobutazone, thielavin B, tiaramide HCl, tiflamizole, timegadine, tolpadol, tryptamid, and ufenamate. The following NSAIDs, designated by company code number (see e.g., Pharmaprojects), may also be used: 480156S, AA861, AD1590, AFP802, AFP860, AI77B, AP504, AU8001, BPPC, BW540C, CHINOIN 127, CN100, EB382, EL508, F1044, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301, and WY41770.

Finally, NSAIDs which may also be used include the salicylates, specifically acetyl salicylic acid and the phenylbutazones, and pharmaceutically acceptable salts thereof.

In addition to indomethacin, other preferred NSAIDs are acetyl salicylic acid, diclofenac, fenbufen, fenoprofen, flurbiprofen, ibuprofen, ketoprofen, naproxen, phenylbutazone, piroxicam, sulindac, and tolmetin. Pharmaceutical compositions and combinations comprising compounds of this invention may also contain inhibitors of the biosynthesis of the leukotrienes such as are disclosed in EP 138,481 (Apr. 24, 1985), EP 115,394 (Aug. 8, 1984), EP 136,893 (Apr. 10, 1985), and EP 140,709 (May 8, 1985), which are hereby incorporated herein by reference.

The compounds of this invention may also be used in combination with leukotriene antagonists such as those disclosed in EP 106,565 (Apr. 25, 1984) and EP 104,885 (Apr. 4, 1984) which are hereby incorporated herein by reference and others known in the art such as those disclosed in EP Application Nos. 56,172 (Jul. 21, 1982) and 61,800 (Jun. 10, 1982); and in U.K. Patent Specification No. 2,058,785 (Apr. 15, 1981), which are hereby incorporated herein by reference.

Pharmaceutical compositions and combinations comprising compounds of this invention may also contain as the second active ingredient, or be used in combination therapy with, prostaglandin antagonists such as those disclosed in EP 11,067 (May 28, 1980) or thromboxane antagonists such as those disclosed in U.S. Pat. No. 4,237,160. They may also contain or be used with histidine decarboxylase inhibitors such as α-fluoromethylhistidine, described in U.S. Pat. No. 4,325,961. The compounds of this invention may also be advantageously combined with an H₁ or H₂-receptor antagonist, such as for instance acetamazole, aminothiadiazoles disclosed in EP 40,696 (Dec. 2, 1981), benadryl, cimetidine, famotidine, framamine, histadyl, phenergan, ranitidine, terfenadine and like compounds, such as those disclosed in U.S. Pat. Nos. 4,283,408; 4,362,736; and 4,394,508. The pharmaceutical compositions may also contain or be used in combination with a K⁺/H⁺ ATPase inhibitor such as omeprazole, disclosed in U.S. Pat. No. 4,255,431, and the like. Compounds of this invention may also be usefully combined with most cell stabilizing agents, such as 1,3-bis(2-carboxychromon-5-yloxy)-2-hydroxypropane and related compounds described in British Patent Specifications 1,144,905 and 1,144,906. Another useful pharmaceutical composition comprises compounds of this invention in combination with serotonin antagonists such as methysergide, the serotonin antagonists described in Nature, 316, 126-131 (1985), and the like. Each of the references referred to in this paragraph is hereby incorporated herein by reference.

Other advantageous pharmaceutical combinations comprise the compounds of this invention in combination with anti-cholinergics such as ipratropium bromide and tiotropium, bronchodilators such as the beta agonist salbutamol, metaproterenol, terbutaline, fenoterol, salmeterol, formoterol and the like, and the anti-asthmatic drugs theophylline, choline theophyllinate and enprofylline, the calcium antagonists nifedipine, diltiazem, nitrendipine, verapamil, nimodipine, felodipine, etc., and the corticosteroids, hydrocortisone, methylprednisolone, betamethasone, dexamethasone, beclomethasone, and the like.

Particularly, for the prophylaxis and treatment of asthma, compounds of this invention can be used in combination with orally inhaled corticosteroids, such as beclomethasone (e.g. QVAR® Inhalation Aerosol), budesonide (e.g. Pulmicort Respules), flunisolide (e.g., AEROBID® and AEROBID®-M Inhaler System), fluticasone (e.g., FLOVENT® DISKUS® inhalation powder, FLOVENT®& HFA Inhalation Aerosol), mometasone (e.g., ASMANEX® TWISTHALER®), and triamcinolone (e.g., AZMACORT® Inhalation Aerosol), and also with inhaled corticosteroid/LABA products such as fluticasone propionate/salmeterol (e.g., ADVAIR DISKUS®). The instant compounds could also be used in combination with leukotriene receptor antagonists such as montelukast (e.g., SINGULAIR®); phosphodiesterase 4 (PDE4) inhibitors such as roflumilast, N-Cyclopropyl-1-[3-(1-oxido-3-pyridinylethynyl)phenyl]-1,4-dihydro[1,8]naphthyridin-4-one-3-carboxamide and the compounds disclosed in PCT Publication WO2003/018579; and Very Late Antigen 4 (VLA4) inhibitors such as the compounds disclosed in U.S. Pat. No. 6,229,011, particularly R411 (N-(2-Chloro-6-methylbenzoyl)-4-[(2,6-dichlorobenzoyl)amino]-L-phenylalanine-2-(diethylamino)ethyl ester which is an ester pro-drug of the active moiety, N-(2-chloro-6-methylbenzoyl)-4-[(2,6-dichlorobenzoyl)amino]-L-phenylalanine), and the compounds disclosed in PCT publication WO2006/023396.

Furthermore, additional active agents such as anti-atherosclerotic agents, anti-diabetes agents, anti-obesity agents and agents used for the treatment of metabolic syndrome, may be used in combination with the compounds of this invention. The additional active agent or agents can be lipid altering compounds such as HMG-CoA reductase inhibitors, or agents having other pharmaceutical activities, or agents that have both lipid-altering effects and other pharmaceutical activities. Examples of HMG-CoA reductase inhibitors useful for this purpose include statins in their lactonized or dihydroxy open acid forms and pharmaceutically acceptable salts and esters thereof, including but not limited to lovastatin (MEVACOR®; see U.S. Pat. No. 4,342,767); simvastatin (ZOCOR®; see U.S. Pat. No. 4,444,784); dihydroxy open-acid simvastatin, particularly the ammonium or calcium salts thereof; pravastatin, particularly the sodium salt thereof (PRAVACHOL®; see U.S. Pat. No. 4,346,227); fluvastatin particularly the sodium salt thereof (LESCOL®; see U.S. Pat. No. 5,354,772); atorvastatin, particularly the calcium salt thereof (LIPITOR®; see U.S. Pat. No. 5,273,995); pitavastatin also referred to as NK-104 (see PCT international publication number WO 97/23200); and rosuvastatin (CRESTOR®); see U.S. Pat. No. 5,260,440). Additional active agents which may be employed in combination with a compound of this invention include but are not limited to HMG-CoA synthase inhibitors; cholesterol absorption inhibitors such as ezetimibe (ZETIA®) which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. No. Re. 37721 and U.S. Pat. No. 5,846,966 as well as a fixed dose combination of ezetimibe and simvastatin (VYTORIN®); HDL-raising agents such as cholesterol ester transfer protein (CETP) inhibitors, for example JTT-705 (Japan Tobacco Company) and torcetrapib (Pfizer); squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT1 and -2; microsomal triglyceride transfer protein (MTP) inhibitors; probucol; niacin; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example troglitazone, pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate and gemfibrozil; PPAR dual α/γ agonists such as muraglitazar; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan and losartan with hydrochlorothiazide; angiotensin converting enzyme inhibitors such as enalapril and captopril; calcium channel blockers such as nifedipine and diltiazam; endothelian antagonists; agents that enhance ABC1 gene expression; FXR and LXR ligands including both inhibitors and agonists; bisphosphonate compounds such as alendronate sodium; and cyclooxygenase-2 inhibitors such as rofecoxib, etoricoxib and celecoxib. Anti-obesity agents can be employed in combination with a compound of this invention including, but not limited to, sibutramine, orlistat, topiramate, naltrexone, bupriopion, phentermine, and phentermine/topiramate combination (QNEXA®); NPY5 antagonists; Acetyl-CoA Carboxylase-1 and -2 (ACC) inhibitors; MCH1R antagonists; and CB1 antagonists/inverse agonists such as those described in WO03/077847 and WO05/000809. Additional anti-diabetes agents which may be employed in combination with a compound of this invention include but are not limited to DPP-4 (dipeptidylpeptidase-4) inhibitors such as sitagliptin (JANUVIA®) and vildagliptin (GALVUS®); sulfonylureas e.g., chlorpropamide, tolazamide, glyburide, glipizide, and glimepiride; biguanides, e.g., metformin; alpha-glucosidase inhibitors e.g., acarbose and miglitol; meglitinides e.g., repaglinide; glucagon-receptor agonists; and glucokinase activators.

Compounds of this invention can be tested using the following assays to determine their mammalian leukotriene biosynthesis inhibiting activity. Representative tested compounds of this invention were shown to be inhibitors of leukotriene biosynthesis, with most having an IC₅₀ less than or equal to 4 μM in the Human 5-Lipoxygenase Enzyme Assay, described below, with preferred compounds tested in this assay having an IC₅₀ less than or equal to 0.100 μM. The representative tested compounds were also shown to have activity as 5-LO inhibitors in the 5-Lipoxygenase Human Whole Blood Assay, described below, with most having an IC₅₀ less than or equal to 4 μM, and preferred compounds having an IC₅₀ of less than or equal to 0.500 μM.

Human 5-Lipoxygenase Enzyme Assay: The activity of 5-lipoxygenase was measured using a spectrophotometric assay and recombinant human 5-lipoxygenase as a source of enzyme. Human 5-lipoxygenase was purified from Sf9 cells infected with the recombinant baculovirus rvHSLO (8-1) containing the coding sequence for human 5-lipoxygenase as described by Percival et al., (Eur. J. Biochem 210, 109-117, 1992). The enzymatic activity was measured using a spectrophotometric assay from the optimal rate of conjugated diene formation (absorbance at 238 nm) using the procedure described in Riendeau et al. (Biochem. Pharmacol. 38, 2313-2321, 1989) with minor modifications. The incubation mixture contained 25 mM potassium phosphate, pH 7.5, 0.1 mM EDTA, 0.3 mM CaCl₂, 24 μg/ml phosphatidylcholine, 0.1 mM ATP, 0.5 mM DTT, 20 μM arachidonic acid (2 μl from a 100-fold solution in ethanol), inhibitor (2 μl aliquot from a 100-fold solution in DMSO) and an aliquot of purified 5-lipoxygenase. Reactions were initiated by the addition of the purified 5-lipoxygenase and the rate of conjugated diene production was followed for 5 minutes at room temperature. The reaction was performed in a Costar UV plate (Cat. #3635) and the absorbance changes at 238 nm were recorded with a Molecular Devices UV/VIS 96 well spectrophotometer (Spectra Max 190) using SOFTmax PRO software. Enzymatic activity was calculated from the optimal rate of the reaction by a linear fit of the increase in absorbance at 238 nm over 36 seconds. When the rate of diene formation is low (<0.01 Absorbance Unit/min) the linear fit is performed over 180 seconds. The results are expressed as percentage of inhibition of the reaction rate relative to controls (typically between 0.001-0.005 Absorbance Unit/min) containing the DMSO vehicle.

5-Lipoxygenase Human Whole Blood Assay: Fresh blood was collected in heparinized tubes by venipuncture from consenting volunteers. These volunteers have no apparent inflammatory conditions and have not taken any nonsteroidal anti-inflammatory drugs for at least 4 days prior to blood collection. Plasma was separated from the blood of each individual volunteer by centrifuging approximately 10 mls of blood. A 50 mM stock solution of the calcium ionophore A23187 (Sigma, St Louis, Mo., USA) in DMSO was diluted 40 fold with each volunteer's plasma to obtain a 1.25 mM working solution. A 250 μl aliquot of each blood was pre-incubated with either 0.5 μl of vehicle (DMSO) or test compounds in DMSO at 37° C. for 15 minutes. This was followed with the addition of 5 μl of either plasma or the 1.25 mM working solution (for each experiment, the blood and plasma was from the same volunteer) resulting in a final concentration of 25 μM of A23187. The blood mixture was incubated at 37° C. for 30 minutes then centrifuged at 1500 g at 4° C. for 10 minutes. The supernatant plasma was collected from all samples and stored at 4° C. All supernatant plasma samples were tested for the production of leukotriene B₄ (LTB₄) using the LTB₄ enzyme immunosorbent assay (EIA) kit from Assay Designs (Ann Arbor, Mich., USA) according to the manufacturer's instructions.

Compounds of this invention may be prepared employing general synthetic procedures known in the art, including methods described in U.S. Pat. No. 5,552,437 and PCT publication WO2004/108720, published Dec. 16, 2004, both publications herein incorporated by reference in their entirety. The synthetic routes outlined in the following methods, reaction schemes and Examples are provided for illustrative purposes only and are not intended to limit the scope of the invention.

Some abbreviations used herein include: Ac=acyl; AIBN=2,2′-azobisisobutyronitrile; CAN=cerium ammonium nitrate; DAST=diethylaminosulfur trifluoride; DBU=1,8-diazabicyclo[5.4.0]undec-7-ene; DCC=1,3-dicyclohexylcarbodiimide; DCM=dichloromethane; DME=ethylene glycol dimethyl ether; DMF=N,N-dimethylformamide; DMSO=dimethyl sulfoxide; EtOH=ethanol; Et₂O=diethyl ether; Et₃N=triethylamine; EtOAc=ethyl acetate; h=hours; HOAc=acetic acid; KHMDS=potassium bis(trimethylsilyl)amide; LAH=lithium aluminum hydride; LDA=lithium diisopropylamide; m-CPBA=3-chloroperoxybenzoic acid; MeOH=methanol; NBS=N-bromosuccinimide; NMO=4-methylmorpholine N-oxide; NMP=1-methyl-2-pyrrolidinone; OTf=trifluoromethanesulfonate=triflate; O-THP=O-tetrahydropyran-2-yl; PPTS=pyridinium p-toluenesulfonate; rt=room temperature; TBAF=tetrabutylammonium fluoride; Tf₂O=triflic anhydride; TFA=trifluoroacetic acid; THF=tetrahydrofuran; TMSCN=trimethylsilyl cyanide; Ph=phenyl; Ac=acetyl.

1,3,4-Oxadiazoles can be prepared according to literature procedures and references cited therein with the appropriate starting material: White, A. D. et al., J. Med. Chem.; 1996, 39, 4382; Futaki, K.; Tosa, S., Chem. Pharm. Bull., 1960, 8, 908; Chem. Abstr., 1966, 64, 3558a.

1,3,4-Thiadiazoles can be prepared according to literature procedures and references cited therein with the appropriate starting material: Werber, G., et al., J. Heterocyclic Chem., 1975, 12, 581; Pandey, V. K., et al., Ind. J. Chem. Sect. B, 2003, 42, 2583; Shaban, M. A. E., et al., Pharmazia, 2003, 58, 6; Miyamoto, K., et al., Chem. Pharm. Bull., 1985, 33, 5126; Yokohama, S., et al., Chem. Pharm. Bull., 1992, 40, 2391; White, A. D., et al., J. Med. Chem., 1996, 39, 4382; Bartels-Keith, J. R., J. Org. Chem., 1977, 42, 3725.

Groups designated as “R” within the methods described below, as well as solvents, temperatures and others reaction conditions may be selected by one of ordinary skill in the art. Functional groups can be either protected or converted to other functional groups. For example, amino groups can be acylated with an acyl chloride or and anhydride with a mild base such as K₂CO₃ or nitrogen base. Esters can be converted to tertiary alcohols or ketones with a Grignard reagent or an alkyllithium reagent.

METHOD A (see scheme below): Compounds 2 to 4 are prepared according to procedures described in U.S. Pat. No. 5,552,437. The 7-methyl group is converted to the mono or dibromide intermediate 5 with NBS and heating in an inert solvent such as CCl₄ in the presence of a radical initiator, such as benzoyl peroxide, AIBN, and light. The monobromide 5 is treated with an excess of NMO at elevated temperatures in a solvent such as dioxane until complete conversion to the aldehyde 6 is observed. Alternatively, the dibromide 5 with AgNO₃ in aqueous dioxane at reflux also affords aldehyde 6. The aldehyde 6 can also be obtained from the dibromide with a hot solution of NH₄OAc in HOAc.

The aldehyde 6 and the amine 7 are refluxed together optionally in the presence of an acid catalyst such as PPTS in a solvent that forms an azeotrope, such as toluene, to yield an imine. This imine is reduced with sodium triacetoxyborohydride or sodium borohydride, or the like, in ethanol or methanol to give amine 8. The imine can be reacted with Grignard reagents or alkyl and aryl lithium reagents at diminished temperatures to give a compound with a substituent alpha to the nitrogen. Amine 8 is also prepared from the reaction of 10 with monobromide 5 to yield 11. The N-acetyl group is removed with a base such as NH₄OH in aqueous THF to yield compound 8. The free NH can be converted to N-alkyl, N-alkanoyl or N-aroyl derivatives with alkyl halides, aliphatic acyl halides or aromatic acyl halides in the presence of a mild base in a solvent such as DCM.

METHOD B (see scheme below): Intermediates 12 (via the N-oxide) and 13 are prepared according to U.S. Pat. No. 5,552,437. Thiol 14 is obtained from the alkaline salt of 2-(trimethylsilyl)ethanethiol which is prepared from a hydride such as NaH in DMF at low ice temperature. The salt thus obtained is treated at room temperature with 13 until disappearance of starting material. Cleavage of the silyl group is effected with a fluoride source such as TBAF at room temperature in THF. To this reaction mixture is added a solution of 15 in acetonitrile, which is then heated to yield compound 16.

METHOD C (see scheme below): By heating an ester 17 with hydrazine, the hydrazide 18 can be obtained. A solution of 18, CS₂ and powdered KOH in ethanol is heated to reflux for 10-18 h and cooled to room temperature. The solvent is removed and the residue dissolved in 25% NH₄OAc. After removal of the solvent, the crude product is purified by flash column chromatography to yield 19. Alkylation with methyl iodide and a mild base such as Cs₂CO₃ followed by oxidation affords intermediate 15.

METHOD D (see scheme below): To a THF solution of 21 at −78° C. is added BuLi followed by chlorotrimethylsilane. The temperature is raised to −20° C., cooled back to −78° C. and BuLi is added followed by a reagent having a carbonyl group to furnish 22.

METHOD E (see scheme below): A solution of alkyl(methylthio)(thioxo)acetate 25, (see: Z. Chem. 1977, vol. 17, 366) and hydrazinecarbodithioic acid potassium salt 23 (see: J. Am. Chem. Soc. 1983, 105, 2287) in EtOH is heated to reflux overnight. After cooling, the reaction mixture is extracted with Et₂O and the solvent removed. The residue is purified by flash column chromatography on silica gel to give intermediate 24.

METHOD F (see scheme below): To a solution of hydrazide 18 and a base such as KHCO₃ in water is added BrCN portionwise. After a brief period of time, the resulting precipitate is filtered and washed with ethyl ether to give intermediate 26.

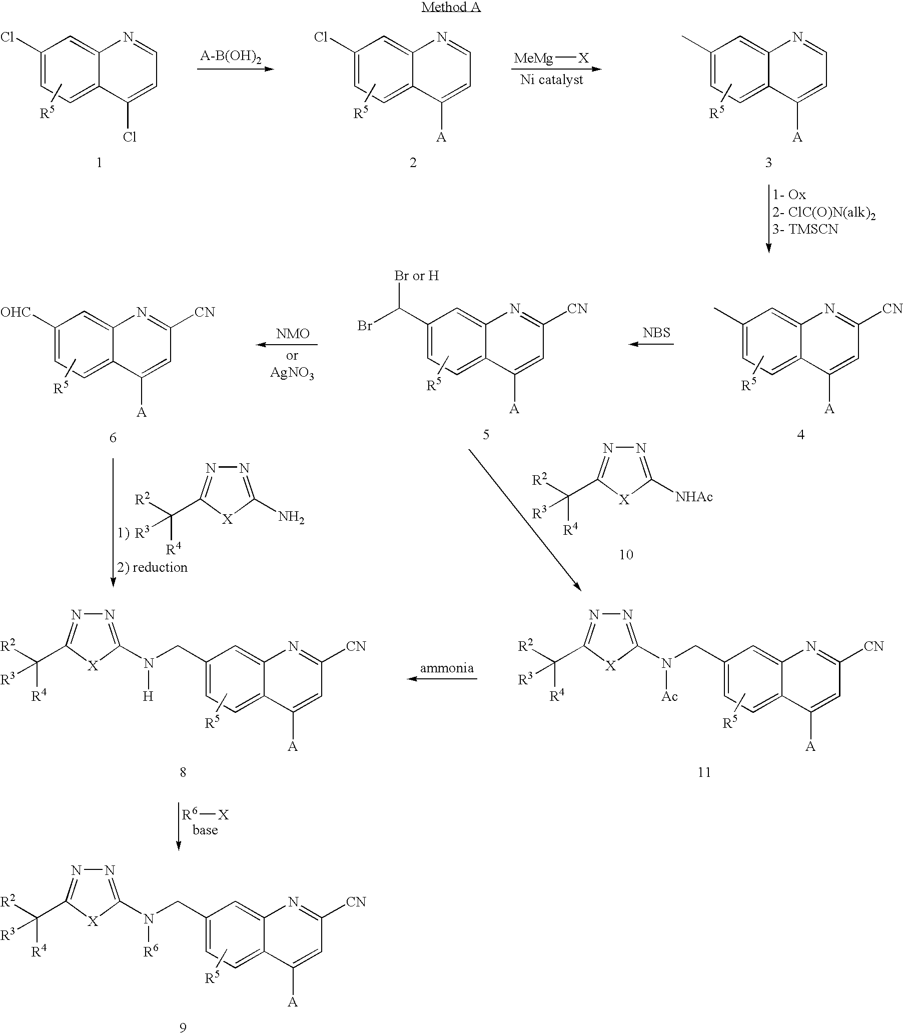

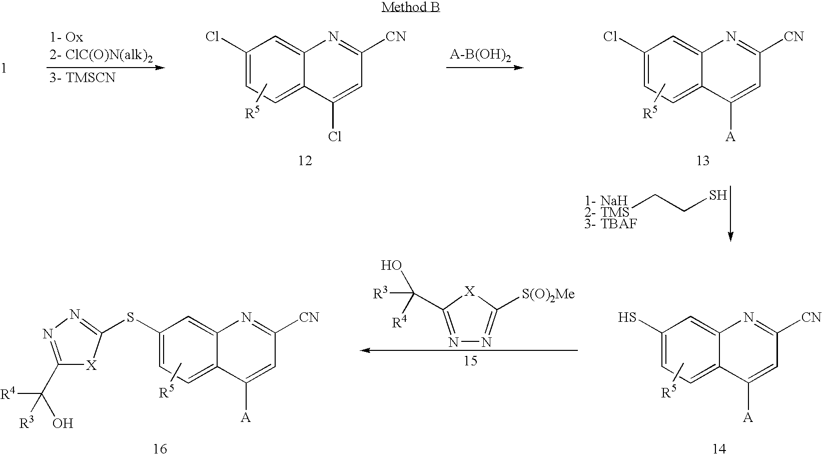

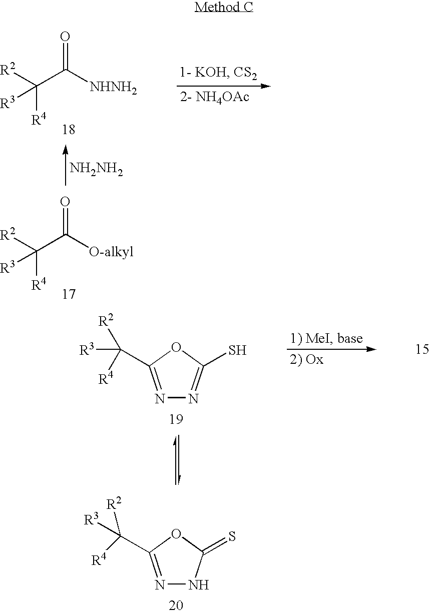

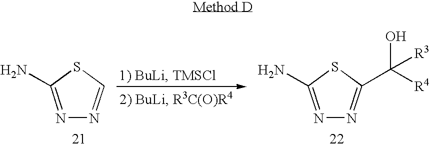

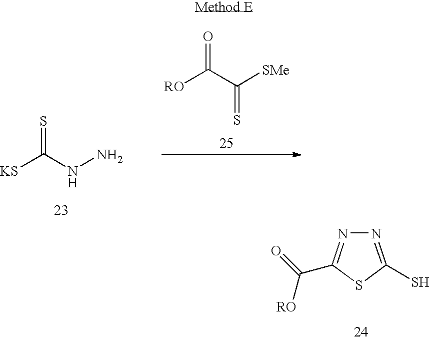

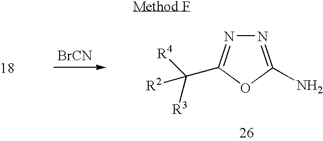

### EXAMPLE 1

### 7-{[5-(1-Ethyl-1-hydroxypropyl)-1,3,4-oxadiazol-2-yl]thio}-4-(3-methylphenyl)quinoline-2-carbonitrile

### Step 1: 3-(5-Mercapto-1,3,4-oxadiazol-2-yl)pentan-3-ol

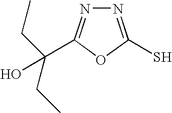

A mixture of methyl 2-ethyl-2-hydroxybutanoate (19.7 g, 134.7 mol) and hydrazine hydrate (14 mL) was heated to 130° C. for 4 h. The mixture was cooled to rt and excess reagent removed under vacuum to yield the hydrazide. To a solution of this hydrazide (3.0 g, 20.5 mmol) in 95% ethanol (80 mL) were added successively crushed potassium hydroxide (1.15 g, 20.5 mmol) and carbon disulfide (1.4 mL, 22.6 mmol). The mixture was stirred at room temperature for 30 min and refluxed for 18 h. The reaction mixture was cooled to room temperature, diluted with toluene (150 mL) and concentrated to dryness. The residue was diluted with 25% aqueous ammonium acetate (100 mL) and concentrated under vacuum to give a mixture of oil and solid. The solid was extracted four times with ethyl acetate by swishing. The organic extracts were concentrated to give an oily residue and purified on silica gel eluting with ethyl acetate to provide the title compound.

### Step 2: 3-[5-(Methylthio)-1,3,4-oxadiazol-2-yl]pentan-3-ol

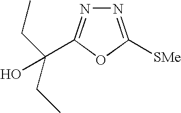

To a solution of 3-(5-mercapto-1,3,4-oxadiazol-2-yl)pentan-3-ol (2.1 g, 10.8 mmol) in DMF (35 mL) were added successively cesium carbonate (7.0 g, 21.6 mmol) and methyl iodide (0.87 mL, 14.0 mmol). The mixture was stirred at room temperature for 75 min and poured into 25% aqueous ammonium acetate. After three extractions with ethyl acetate, the combined organic extracts were washed three times with water, dried (MgSO₄) and concentrated to give the crude compound. Flash chromatography of the residue on silica gel (eluting with 9:1 dichloromethane-acetone) provided the title compound.

### Step 3: 3-[5-(Methylsulfonyl)-1,3,4-oxadiazol-2-yl]pentan-3-ol

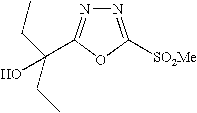

To a solution of 3-[5-(methylthio)-1,3,4-oxadiazol-2-yl]pentan-3-ol (1.9 g, 9.3 mmol) in dichloromethane (80 mL) was added in one portion 80% 3-chloroperoxybenzoic acid (8.0 g, 37.2 mmol) and stirred at room temperature for 18 h. Solid calcium hydroxide (3.0 g, 42 mmol) was added and stirring was continued for 30 min. The mixture was filtered through celite, rinsed with dichloromethane and liquors evaporated to dryness to give a crude solid. Flash chromatography of the residue on silica gel (eluting with 9:1 dichloromethane/acetone) provided the title compound. ¹NMR (400 MHz, acetone-d₆): δ 4.98 (s, 1H), 3.60 (s, 3H), 1.95-2.10 (m, 4H), 0.90 (t, 6H).

### Step 4: 4,7-Dichloroquinoline 1-oxide

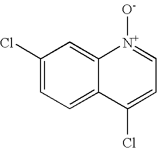

To a suspension of 4,7-dichloroquinoline (19.8 g, 100 mmol) in dichloromethane (1500 mL) was added 70% 3-chloroperoxybenzoic acid (21.7 g, 110 mmol) and stirred at room temperature for 18 h. Solid calcium hydroxide (22.2 g, 300 mmol) was added and 15 min later the mixture was filtered through celite, rinsed with dichloromethane and the liquors evaporated to dryness to give the title compound. The compound was used as such for the next step. ¹NMR (400 MHz, acetone-d₆): δ 8.65 (s, 1H), 8.50 (d, 1H), 8.28 (d, 1H), 7.9 (dd, 1H), 7.65 (d, 1H).

### Step 5: 4,7-Dichloroquinoline-2-carbonitrile

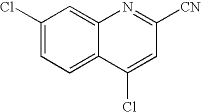

To a solution of 4,7-dichloroquinoline 1-oxide (19.5 g, 91 mmol) in 1,2-dichloroethane (1000 mL) was added trimethylsilyl cyanide (24.3 mL, 182 mmol) followed by dimethylcarbamoyl chloride (16.8 mL, 182 mmol) and stirred at 60° C. for 8 h. The solution was quenched slowly with 1M sodium carbonate (250 mL), stirred at room temperature for 10 min, diluted with water and extracted two times with dichloromethane. The combined organic extracts were washed two times with water, dried (MgSO₄) and concentrated to give the crude compound. Flash chromatography of the residue on silica gel (eluting with hexane-ethyl acetate-dichloromethane, 10:2:1) provided the title compound. ¹NMR (400 MHz, acetone-d₆): δ 8.40 (d, 1H), 8.28 (s, 1H), 8.25 (s, 1H), 7.98 (dd, 1H).

### Step 6: 7-Chloro-4-(3-methylphenyl)quinoline-2-carbonitrile

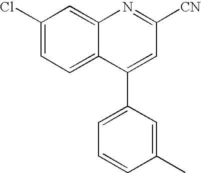

To a solution of 4,7-dichloroquinoline-2-carbonitrile (2.23 g, 10 mmol) in DME (75 mL) were added successively tetrakis(triphenylphosphine)palladium(0) (0.578 g, 10.5 mmol), 3-methylbenzeneboronic acid (1.63 g, 12 mmol) and cesium fluoride (3.8 g, 25 mmol). The mixture was stirred at reflux for 12 h, cooled to room temperature, diluted with water and extracted three times with ethyl acetate. The combined organic extracts were washed with water, dried (MgSO₄) and concentrated to give the crude compound. Flash chromatography of the residue on silica gel (eluting with toluene) provided the title compound. ¹NMR (500 MHz, acetone-d₆): δ 8.20 (d, 1H), 8.08 (d, 1H), 7.92 (s, 1H), 7.78 (dd, 1H), 7.53 (t, 1H), 7.43-7.48 (m, 3H), 2.47 (s, 3H).

### Step 7: 4-(3-Methylphenyl)-7-{[2-trimethylsilyl)ethyl]thio}quinoline-2-carbonitrile

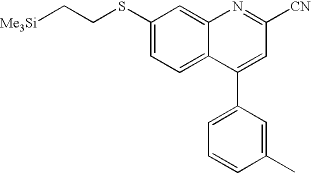

To a suspension of sodium hydride 60% dispersion in oil (0.120 g, 3.0 mmol) in DMF (3 mL) at 0° C. was added 2-(trimethylsilyl)ethanethiol (0.26 mL, 1.6 mmol). The mixture was stirred at 0° C. for 10 min and the cooling bath was removed. To this suspension was added a solution of 7-chloro-4-(3-methylphenyl)quinoline-2-carbonitrile (0.279 g, 1.0 mmol) in DMF (3 mL) and was stirred at room temperature for 40 min. The reaction mixture was quenched with aqueous saturated ammonium chloride and extracted three times with ethyl acetate. The combined organic extracts were washed with water, dried (MgSO₄) and concentrated to give the crude compound. Flash chromatography of the residue on silica gel (eluting with dichloromethane-hexane, 2:1) provided the title compound. ¹NMR (500 MHz, acetone-d₆): δ 8.02 (s, 1H), 8.00 (s, 1H), 7.78 (s, 1H), 7.60 (dd, 1H), 7.52 (t, 1H), 7.38-7.48 (m, 3H), 3.25-3.35 (m, 2H), 2.45 (s, 3H), 1.05-1.12 (m, 2H), 0.15 (s, 9H).

### Step 8: 7-{[5-(1-Ethyl-1-hydroxypropyl)-1,3,4-oxadiazol-2-yl]thio}-4-(3-methylphenyl)quinoline-2-carbonitrile

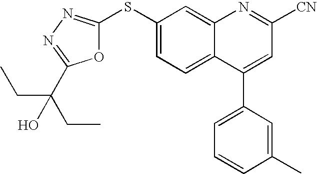

To a solution of 4-(3-methylphenyl)-7-{[2-trimethylsilyl)ethyl]thio}quinoline-2-carbonitrile (0.304 g, 0.81 mmol) in THF (4 mL) was added a 1M solution of tetrabutylammonium fluoride in THF (1.6 mL, 1.6 mmol). After 4 h of stirring, a solution of 3-[5-(methylsulfonyl)-1,3,4-oxadiazol-2-yl]pentan-3-ol (0.246 g, 1.05 mmol) obtained from Step 3 in acetonitrile (4 mL) was added and heated at 60° C. for 1.5 h. The reaction mixture was quenched with aqueous saturated ammonium chloride and extracted three times with ethyl acetate. The combined organic extracts were washed with water, dried (MgSO₄) and concentrated to give the crude compound. Flash chromatography of the residue on silica gel (eluting with hexane-ethyl acetate, 60:40) provided the title compound. ¹NMR (500 MHz, acetone-d₆): δ 8.40 (s, 1H), 7.95 (s, 1H), 7.85 (dd, 1H), 7.55 (t, 1H), 7.40-7.50 (m, 3H), 4.75 (s, 1H), 2.50 (s, 3H), 1.90-2.02 (m, 4H), 0.90 (t, 6H). Mass spectrum: 431 (M+1).

### EXAMPLE 2

### 4-Phenyl-7-{[(5-propionyl-1,3,4-oxadiazol-2-yl)amino]methyl}quinoline-2-carbonitrile

### Step 1: 1-(5-Amino-1,3,4-oxadiazol-2-yl)propan-1-one

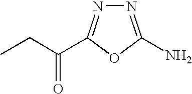

To a solution of ethyl 5-amino-1,3,4-oxadiazol-2-carboxylate (Chem. Abstract, 1966, 64, 3558a) (0.8 g, 5.1 mmol) in THF (25 mL) at −78° C. was added IM ethylmagnesium bromide (15.3 mL, 15.3 mmol) and brought to rt overnight. The reaction mixture was poured into 25% aqueous ammonium acetate and extracted three times with ethyl acetate. The combined organic layers were washed with brine, dried over anhydrous magnesium sulfate and concentrated under reduced pressure. Flash column chromatography on silica gel using 30% ethyl acetate in hexane gave the desire ketone and the corresponding tertiary alcohol. ¹NMR (400 MHz, acetone-d₆): δ 7.1 (br, 2H), 3.0 (q, 2H), 1.16 (t, 3H).

### Step 2: 7-Chloro-4-phenylquinoline

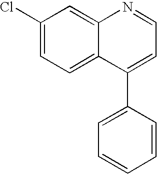

A mixture of 4,7-dichloroquinoline (12.5 g, 63.1 mmol), phenylboronic acid (9.63 g, 79 mmol) and cesium fluoride (24 g, 158 mmol) in 1,2-dimethoxyethane (300 mL) was degassed and purged three times with nitrogen gas before the addition of the tetrakis(triphenylphosphine) palladium(0) (3.64 g, 3.15 mmol). The resultant mixture was then stirred at reflux overnight. After cooling, the reaction was filtered over celite and washed with dichloromethane. After evaporation under reduced pressure, the residue was purified by column chromatography (eluting with hexane/EtOAc, 95:5) to yield the title compound. ¹H NMR (400 MHz, acetone d₆): δ 9.0 (d, 1H), 8.15 (d, 1H), 7.95 (d, 1H), 7.65-7.55 (m, 6H), 7.5 (d, 1H).

### Step 3: 7-Methyl-4-phenylquinoline

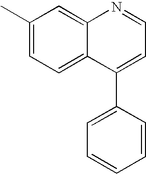

To a solution of 7-chloro-4-phenylquinoline (10.0 g, 41.7 mmol) and Ni(dppp)₂Cl₂ (2.26 g, 4.17 mmol) in ether (200 mL) was added dropwise 3.0 M MeMgBr (24.3 mL, 73 mmol) and heated at reflux for 3 h. The reaction mixture was quenched with saturated aqueous NH₄Cl solution and extracted with ether. The combined organic layer was washed with water and brine and then dried over anhydrous MgSO₄. The solvent was removed under reduced pressure, and the resulting crude product was used in the next step without further purification. ¹H NMR (400 MHz, acetone-d₆): δ 8.9 (d, 1H), 7.93 (bs, 1H), 7.82 (d, 1H), 7.52-7.62 (m, 5H), 7.43 (dd, 1H), 7.35 (d, 1H), 2.60 (S, 3H).

### Step 4: 7-Methyl-4-phenylquinoline-2-carbonitrile

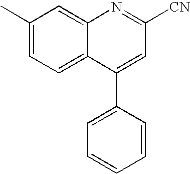

A solution of 7-methyl-4-phenylquinoline (10.0 g, 41.7 mmol) and m-CPBA (13.5 g, 54.8 mmol) in chloroform (230 mL) was stirred at room temperature for 3 h. The reaction was quenched with saturated aqueous NaHCO₃ solution and extracted twice with dichloromethane. The combined organic layers were washed with water, brine, and dried over anhydrous MgSO₄. The solvent was removed under reduced pressure and the resulting crude product was dissolved in chloroform (220 mL). N,N-Dimethylcarbamoyl chloride (8.44 mL, 91.4 mmol) and trimethylsilyl cyanide (11.5 mL, 91.4 mmol) were added to this solution and stirred at room temperature for 2 d. Saturated aqueous NaHCO₃ solution was added and stirred for 30 min. The organic layer was removed, and the aqueous layer was extracted with dichloromethane. The combined organic layers were washed with water, brine, and dried over anhydrous MgSO₄. The solvent was removed under reduced pressure and the resulting crude product was used in the next step without further purification. ¹H NMR (400 MHz, acetone-d₆): δ 8.01 (s, 1H), 7.92 (d, 1H), 7.80 (s, 1H), 7.60-7.67 (m, 6H), 2.63 (s, 3H).

### Step 5: 7-(Bromomethyl)-4-phenylquinoline-2-carbonitrile

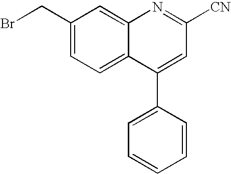

A solution of 7-methyl-4-phenylquinoline-2-carbonitrile (11.2 g, 41.7 mmol), N-bromosuccinimide (12.2 g, 68.6 mmol) and AIBN (2,2′-azobisisobutyronitrile) (200 mg) in carbon tetrachloride (200 mL) was heated at reflux overnight. The reaction was cooled to ambient temperature and the solid residue was filtered. The filtrate was concentrated under reduced pressure. The crude product was purified by flash column chromatography (eluting with 5% ethyl acetate in hexane) to give the title compound. ¹H NMR (400 MHz, acetone-d₆): δ 8.30 (s, 1H), 8.06 (d, 1H), 7.90 (s, 1H), 7.87 (d, 1H), 7.6-7.7 (m, 5H), 4.95 (s, 2H).

### Step 6: 4-Phenyl-7-{[(5-propionyl-1,3,4-oxadiazol-2-yl)amino]methyl}quinoline-2-carbonitrile

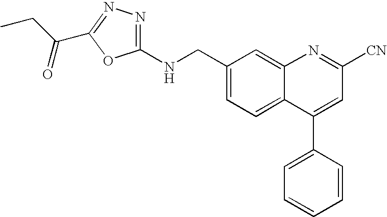

A solution of 1-(5-amino-1,3,4-oxadiazol-2-yl)propan-1-one (0.070 g, 0.5 mmol), 7-(bromomethyl)-4-phenylquinoline-2-carbonitrile (0.242 g, 0.75 mmol) and N,N-diisopropylethylamine (261 μL, 1.5 mmol) in DMF (1.5 mL) was heated in a sealed tube at 150° C. for 10 min in a microwave apparatus. The reaction was concentrated under reduced pressure and the crude product purified on silica gel (eluting with dichloromethane-ethyl acetate-hexane, 1:3:6) to yield the desired product. ¹H NMR (acetone-d₆): δ 8.22 (s, 1H), 8.03 (d, 2H), 7.84-7.89 (m, 2H), 7.60-7.68 (m, 5H), 4.94 (s, 2H), 3.0 (q, 2H), 1.16 (t, 3H). Mass spectrum: 384 (M+1).

### EXAMPLE 3

### 7-[({5-[Dicyclopropyl(hydroxy)methyl]-1,3,4-oxadiazol-2-yl}amino)methyl]-4-(3-fluorophenyl)quinoline-2-carbonitrile

### Step 1: (5-Amino-1,3,4-oxadiazol-2-yl)(dicyclopropyl)methanol

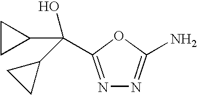

To a solution of 0.5M cyclopropylmagnesium bromide in THF (500 mL, 250 mmol) cooled at −78° C. was added dropwise a solution of ethyl 5-amino-1,3,4-oxadiazol-2-carboxylate (5.0 g, 31.8 mmol) in THF (250 mL, 0.127M). The temperature was then slowly raised to rt. The solution was quenched with aqueous NH₄Cl and the isolated organic layer was washed with brine, dried over MgSO₄, filtered and concentrated under reduced pressure. The residue obtained was purified by column chromatography (eluting with ether-toluene-EtOAc-MeOH, 80:20:0:0 to 0:0:100:0 to 0:0:95:5) to yield the title compound. ¹H NMR (400 MHz, acetone-d₆): δ 6.25, (bs, 2H), 4.1 (s, 1H), 1.35-1.25, (m, 2H), 0.65-0.50 (m, 4H) 0.5-0.3 (m, 4H).

### Step 2: 7-Chloro-4-(3-fluorophenyl)quinoline

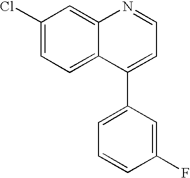

To a solution of 4,7-dichloroquinoline (13.8 g, 69.7 mmol) in 1,2-dimethoxyethane (300 mL, 0.232M), 3-fluorophenylboronic acid (12.17 g, 87 mmol) and cesium fluoride (26.4 g, 174 mmol) were added. The mixture was then degassed and purged three times with nitrogen gas before the addition of tetrakis(triphenylphosphine)palladium(0) (4.02 g, 3.48 mmol) and stirred at reflux overnight. After cooling, the crude reaction mixture was filtered over celite and washed with dichloromethane. The organic layer was removed under reduced pressure and the residue was purified by column chromatography (eluting with hexane-EtOAc, 95:5) to yield the title compound. ¹H NMR (400 MHz, acetone-d₆): δ 9.0 (d, 1H), 8.15 (s, 1H), 7.95 (d, 1H), 7.7-7.55 (m, 2H), 7.5 (d, 1H), 7.45-7.3 (m, 3H).

### Step 3: 4-(3-Fluorophenyl)-7-methylquinoline

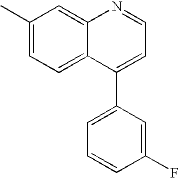

To a solution of 7-chloro-4-(3-fluorophenyl)quinoline (16.2 g, 62.9 mmol) and (1,3-bis(diphenylphosphino)propane)-dichloronickel(II) (3.41 g, 6.29 mmol) in ether (150 mL, 0.419M), methylmagnesium bromide (36.7 mL, 110 mmol) was added slowly. The mixture was then stirred at reflux for 3 h. After cooling to 0° C., a solution of NH₄Cl was added. The aqueous phase was extracted with ether and the organic layers combined. The combined organic phases were washed with water, brine, dried over MgSO₄, filtered and concentrated to yield the title compound. The crude residue was used without further purification in the next step. ¹H NMR (400 MHz, acetone-d₆): δ 8.9 (d, 1H), 7.95 (s, 1H), 7.8 (d, 1H), 7.65 (q, 1H), 7.45 (d, 1H), 7.4-7.35 (m, 4H).

### Step 4: 4-(3-Fluorophenyl)-7-methylquinoline 1-oxide

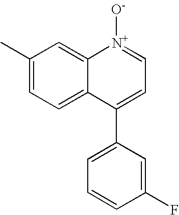

To a solution of 4-(3-fluorophenyl)-7-methylquinoline (5.0 g, 21.1 mmol) in chloroform (200 mL) was added 3-chloroperoxybenzoic acid (5.46 g, 25.3 mmol) and the reaction mixture was stirred overnight at rt. Calcium hydroxide (3.91 g, 52.7 mmol) was added and 15 min later the mixture was filtered. Removed of the solvent under vacuum gave the title compound. ¹H NMR (400 MHz, acetone-d₆): δ 8.6 (s, 2H), 7.85 (d, 1H), 7.7-7.5 (m, 2H), 7.45-7.25 (m, 4H), 2.6 (s, 3H).

### Step 5: 4-(3-Fluorophenyl)-7-methylquinoline-2-carbonitrile

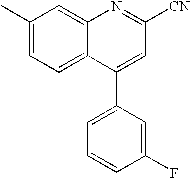

A mixture of 4-(3-fluorophenyl)-7-methylquinoline 1-oxide (5.34 g, 21.1 mmol), trimethylsilylcyanide (5.66 mL, 42.2 mmol) and dimethylcarbamoyl chloride (3.88 mL, 42.2 mmol) in chloroform (150 mL) was stirred at rt overnight. A saturated NaHCO₃ solution was added and the mixture was stirred for 5 min. The isolated aqueous layer was extracted with CH₂Cl₂ and the organic extract was washed with brine, dried over MgSO₄, filtered and concentrated under reduced pressure. The crude residue was purified by column chromatography on silica gel (eluting with CH₂Cl₂) to yield the title compound. ¹H NMR (400 MHz, acetone-d₆): δ 8.0 (s, 1H), 7.9 (d, 1H), 7.8 (s, 1H), 7.75-7.6 (m, 2H), 7.5-7.3 (m, 3H), 2.6 (s, 3H).

### Step 6: 7-(Dibromomethyl)-4-(3-fluorophenyl)quinoline-2-carbonitrile

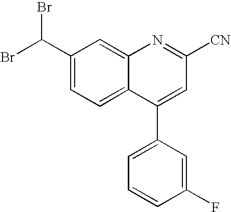

A solution of 4-(3-fluorophenyl)-7-methylquinoline-2-carbonitrile (2.0 g, 7.6 mmol), N-bromosuccinimide (1.90 g, 10.67 mmol) and AIBN (50.1 mg, 0.305 mmol) in CCl₄ (40 mL) was stirred at reflux overnight. After cooling, the mixture was filtered and the solid collected was rinsed with CCl₄. The filtrate was concentrated under reduced pressure and the crude residue obtained was suspended overnight in a 10:1 mixture of hexanes and Et₂O to yield the title compound. ¹H NMR (400 MHz, acetone-d₆): δ 8.4 (s, 1H), 8.15 (s, 2H), 8.0 (s, 1H), 7.8-7.65 (m, 1H), 7.55, 7.45 (m, 3H), 7.4 (t, 1H).

### Step 7: 4-(3-Fluorophenyl)-7-formylquinoline-2-carbonitrile

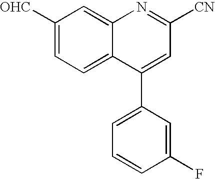

To a solution of 7-(dibromomethyl)-4-(3-fluorophenyl)quinoline-2-carbonitrile (2.02 g, 4.81 mmol) in 1,4-dioxane (75 mL) was added a solution of silver nitrate (3.27 g, 19.24 mmol) in 30 mL of water, and the reaction mixture stirred 1 h at reflux. After cooling to rt, the mixture was diluted with EtOAc and filtered. The solid was washed with EtOAc and the organic phases combined. The filtrate was washed twice with water and the combined aqueous layers were back-extracted twice with EtOAc. The combined organic extracts were washed with brine, dried over MgSO₄, filtered and concentrated. The crude residue obtained was purified by column chromatography (eluting with acetone-CH₂Cl₂-toluene, 10:40:50) to yield the title compound. ¹H NMR (400 MHz, acetone-d₆): δ 10.4 (s, 1H), 8.8 (s, 1H), 8.2 (s, 2H), 8.1 (s, 1H), 7.75-7.65 (m, 1H), 7.55-7.45 (m, 2H), 7.45-7.35 (m, 1H).

### Step 8: 7-[({5-Dicyclopropyl(hydroxy)methyl-1,3,4-oxadiazol-2-yl}amino)methyl]-4-(3-fluorophenyl)quinoline-2-carbonitrile

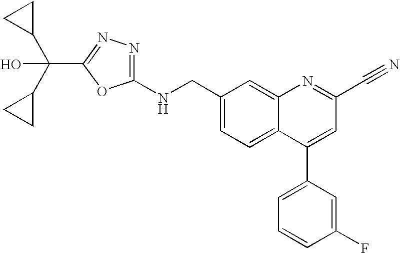

A mixture of 5-amino-1,3,4-oxadiazol-2-yl)(dicyclopropyl)methanol (0.265 g, 1.36 mmol), 4-(3-fluorophenyl)-7-formylquinoline-2-carbonitrile (0.300 g, 1.086 mmol) and PPTS (0.027 g, 0.11 mmol) in benzene (2.5 mL) was stirred overnight at reflux with a Dean-Stark trap. After cooling, the mixture was diluted in THF (5 mL) and sodium triacetoxyborohydride (1.151 g, 5.4 mmol) was added and brought to 45° C. for 3 h. The reaction mixture was quenched with a saturated NaHCO₃ solution and partitioned between EtOAc and water. The organic layer was dried over MgSO₄, filtered and concentrated. The crude residue obtained was purified by column chromatography (eluting with EtOH—CHCl₃, 5:95) to yield the title compound. ¹H NMR (400 MHz, acetone-d₆): δ 8.25 (s, 1H), 8.0 (d, 1H), 7.95-7.85 (m, 2H), 7.75-7.6 (m, 1H), 7.5-7.3 (m, 4H), 4.8 (d, 2H), 4.15 (s, 1H), 1.4-1.25 (m, 2H), 0.65-0.5 (m, 4H), 0.5-0.3 (m, 4H). Mass spectrum: 454 (M−1).

### EXAMPLE 4

### N-{[2-Cyano-4-(3-fluorophenyl)quinolin-7-yl]methyl}-N-[5-(1-ethyl-1-hydroxypropyl)-1,3,4-oxadiazol-2-yl]acetamide

### Step 1: N-[5-(1-Ethyl-1-hydroxypropyl)-1,3,4-oxadiazol-2-yl]acetamide

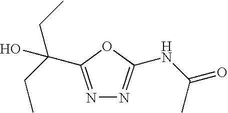

To a solution of 3-(5-amino-1,3,4-oxadiazol-2-yl)pentan-3-ol (1.17 g, 6.8 mol) and N,N-diisopropylethylamine (1.94 g, 15.0 mmol) in 50 mL of THF at 0° C. was added acetyl chloride (0.591 g, 7.5 mmol). After 1.5 h of stirring, the mixture was partitioned between EtOAc and water, the organic layer separated, dried, and the solvent removed. Trituration with Et₂O-hexane (1:1) and filtration of the solid yielded the title compound. ¹H NMR (400 MHz, acetone-d₆): δ 7.65 (bs, 1H), 4.63 (s, 1H), 2.40 (s, 3H), 1.8-1.9 (m, 4H), 0.9-1.0 (m, 6H).

### Step 2: 7-(Bromomethyl)-4-(3-fluorophenyl)quinoline-2-carbonitrile

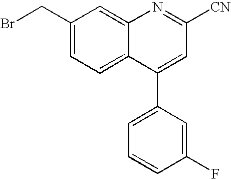

A solution of 4-(3-fluorophenyl)-7-methylquinoline-2-carbonitrile (0.425 g, 1.6 mmol) (prepared as described in Example 2), N-bromosuccinimide (0.317 g, 1.8 mmol), and AIBN (13 mg, 0.08 mmol) in CCl₄ (40 mL) was stirred at reflux for 2.5 h. After cooling, the mixture was filtered and the solid collected was rinsed with CH₂Cl₂. The filtrate was concentrated under reduced pressure and the crude residue obtained was suspended in ether with stirring for 2 h. The solid was filtered to give a mixture of mono- and dibromo adduct (10:3) and used as such in Step 3.

### Step 3: N-{[2-Cyano-4-(3-fluorophenyl)quinolin-7-yl]methyl}-N-[5-(1-ethyl-1-hydroxypropyl)-1,3,4-oxadiazol-2-yl]acetamide

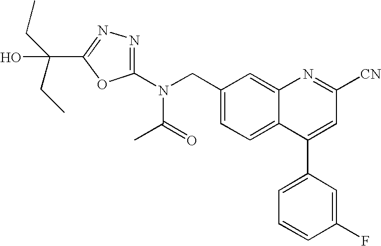

7-(Bromomethyl)-4-(3-fluorophenyl)quinoline-2-carbonitrile (0.220 g, 0.65 mmol), N-[5-(1-ethyl-1-hydroxypropyl)-1,3,4-oxadiazol-2-yl]acetamide (0.110 g, 0.52 mmol), K₂CO₃ (0.214 g, 1.5 mmol) in DMF (5 mL) are stirred for 4 h at rt. The reaction mixture was quenched with aqueous NH₄Cl and EtOAc was added. The organic layer was separated, dried and the solvent removed. Purification on silica gel (eluting with EtOAc-hexane, 2:1) gave the title compound. ¹H NMR (400 MHz, acetone-d₆): δ 8.15 (s, 1H), 8.02 (d, 1H), 7.93 (s, 1H), 7.80 (d, 1H), 7.68 (m, 1H), 7.35-7.58 (m, 3H), 5.37 (s, 2H), 4.62 (s, 1H), 2.51 (s, 3H), 1.89 (m, 4H), 0.81 (t, 3H). Mass spectrum: 474 (M+1).

### EXAMPLE 5

### 4-(4-Fluorophenyl)-7-[({5-[1-hydroxy-1-(trifluoromethyl)propyl]-1,3,4-oxadiazol-2-yl}amino)methyl]quinoline-2-carbonitrile

### Step 1: Ethyl 2-hydroxy-2-(trifluoromethyl)butanoate

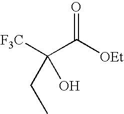

To a −78° C. solution of ethyl trifluoropyruvate (129.0 g 758 mmol) in ether was added dropwise within 90 min a solution of EtMgBr (3.0 M in ether, 252 mL). The solution was cooled over 1 h to about −10° C. and poured over 2 L of saturated NH₄Cl. The layers were separated and the aqueous phase extracted with ether (3×500 mL). The organic phases were combined, dried over MgSO₄ and the solvent removed. Distillation at 50-65° C. (30 mm Hg) gave the title compound. ¹H NMR (400 MHz, acetone-d₆): δ 5.4 (s, 1H), 4.35 (q, 2H), 2.07 (m, 1H), 1.83 (m, 1H), 1.3 (t, 3H) and 0.93 (t, 3H).

### Step 2: 2-Hydroxy-2-(trifluoromethyl)butanohydrazide

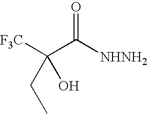

The ethyl ester from Step 1 (50.04 g, 250 mmol) and hydrazine hydrate (25.03 g, 50 mmol) were heated at 80° C. for 18 h. The excess hydrazine was removed under vacuum and the crude product was filtered through a pad of silica gel with EtOAc-Hexane (about 3 L) to furnish the title compound. ¹H NMR (400 MHz, acetone-d₆): δ 9.7 (s, 1H), 6.10 (s, 1H), 2.25 (m, 1H), 1.85 (m, 1H) and 0.95 t, (3H).

### Step 3: 2-(5-Amino-1,3,4-oxadiazol-2-yl)-1,1,1-trifluorobutan-2-ol

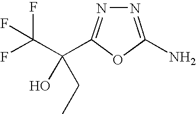

To the hydrazide (34.07 g, 183 mmol) from Step 2 in 275 mL of water was added KHCO₃ (18.33 g, 183 mmol) followed by BrCN (19.39 g, 183 mmol) portionwise. After 3 h, the white solid was filtered, washed with cold water, and dried to afford the title compound. ¹H NMR (400 MHz, acetone-d₆): δ 6.54 (s, 2H), 6.01 (s, 1H), 2.22 (m, 1H), 2.08 (m, 1H) and 0.99 (m, 3H).

### Step 4: 7-Chloro-4-(4-fluorophenyl)quinoline

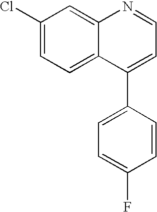

p-Fluorophenylmagnesium bromide (17.6 mmol) was added over 20 min to a solution of 4,7-dichloroquinoline (3.17 g, 16.0 mmol) and Pd(PPh₃)₄ (0.65 g, 0.56 mmol) in 40 mL of THF at rt. After 2 h, more p-fluorophenylmagnesium bromide (4.8 mmol) was added, and the reaction was stirred at rt overnight. The solution was quenched with aqueous NH₄Cl and diluted with EtOAc and water. The organic layer was separated, washed with brine, dried and the solvent removed. Purification on silica gel (eluting with EtOAc-hexane, 1:5) gave the title compound. ¹H NMR (400 MHz, CDCl₃): δ 8.95 (d, 1H), 8.23 (s, 1H), 7.83 (d, 1H), 7.49 (m, 3H), 7.36 (d, 1H), 7.23 (m, 2H).

### Step 5: 4-(4-Fluorophenyl)-7-methylquinoline

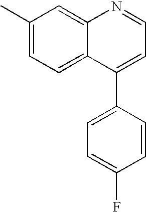

Methylmagnesium bromide (3.0M in ether, 36.4 mmol) was added to 7-chloro-4-(4-fluorophenyl)quinoline (8.52 g, 33.1 mmol) and Ni(dppp)Cl₂ (1.80 g) in 120 mL of THF at rt. Additional methylmagnesium bromide (3.6 mL) was added. After 30 min, the reaction mixture was quenched with aqueous NH₄Cl and diluted with EtOAc and water. The organic layer was separated, washed with brine, dried and the solvent removed. Purification on silica gel (eluting with EtOAc-hexane, 1:5 to 1:3) gave the title compound. ¹H NMR (400 MHz, CDCl₃): δ 8.91 (d, 1H), 7.98 (s, 1H), 7.77 (d, 1H), 7.50 (m, 2H), 7.36 (d, 1H), 7.27 (m, 3H), 2.60 (s, 3H).

### Step 6: 4-(4-Fluorophenyl)-7-methylquinoline-2-carbonitrile

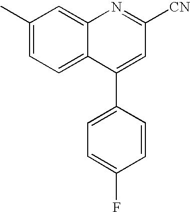

4-(4-Fluorophenyl)-7-methylquinoline (4.48 g, 19.3 mmol) was treated according to Step 4 of Example 2 to yield the title compound. ¹H NMR (400 MHz, CDCl₃): δ 8.04 (s, 1H), 7.83 (d, 1H), 7.49 (s, 1H), 7.48 (m, 3H), 7.28 (m, 2H), 2.62 (s, 3H).

### Step 7: 4-(4-Fluorophenyl-7-formylquinoline-2-carbonitrile

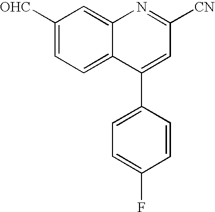

Following the conditions described in Steps 6 and 7 of Example 3,4-(4-fluorophenyl)-7-methylquinoline-2-carbonitrile (3.69 g, 14.1 mmol) afforded the title compound. ¹H NMR (400 MHz, CDCl₃): δ 10.32 (s, 1H), 8.72 (s, 1H), 8.17 (d, 1H), 8.08 (d, 1H), 7.77 (s, 1H), 7.51 (m, 2H), 7.32 (m, 2H).

### Step 8: 4-(4-Fluorophenyl)-7-[({5-[1-hydroxy-1-(trifluoromethyl)propyl]-1,3,4-oxadiazol-2-yl}amino)methyl]quinoline-2-carbonitrile

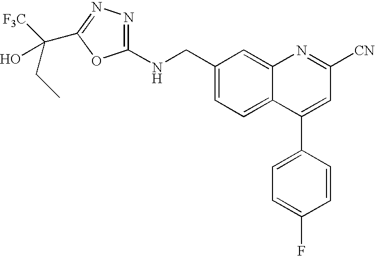

Following the conditions of Step 8 of Example 3, [4-(4-fluorophenyl)-7-formylquinoline-2-carbonitrile (0.166 g, 0.6 mmol) and 2-(5-amino-1,3,4-oxadiazol-2-yl)-1,1,1-trifluorobutan-2-ol (0.127 g, 0.6 mmol) afforded the title compound. ¹H NMR (400 MHz, CDCl₃): δ 8.22 (s, 1H), 7.98 (d, 1H), 7.71 (d, 1H), 7.66 (s, 1H), 7.50 (m, 2H), 7.30 (m, 2H), 6.30 (bs, 1H), 4.87 (s, 2H), 2.15 (m, 2H), 0.93 (t, 3H). Mass spectrum: 472 (M+1).

### EXAMPLE 6

### 7-({[5-(1-Ethyl-1-hydroxypropyl)-1,3,4-thiadiazol-2-yl]amino}methyl)-4-phenylquinoline-2-carbonitrile

### Step 1: Ethyl 5-amino-1,3,4-thiadiazole-2-carboxylate

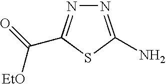

A mixture of ethyl chloro(oxo)acetate (0.5 g, 3.68 mmol) and hydrazinecarbothioamide (0.335 g, 3.68 mmol) was heated to 160° C. for 5 h. The solvent was removed under reduce pressure and the crude purified on silica gel (eluting with 3% methanol in DCM) to give the title product. ¹H NMR (400 MHz, acetone-d₆): δ 7.28 (bs, 1H), 9.67 (bs, 1H), 4.39 (q, 2H), 1.38 (t, 3H).

### Step 2: 3-(5-Amino-1,3,4-thiadiazol-2-yl)pentan-3-ol

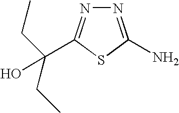

To a solution of ethyl 5-amino-1,3,4-thiadiazole-2-carboxylate (0.95 g, 5.5 mmol) in 18 mL of THF at 0° C. was added slowly a 3.0M solution of EtMgBr (14.6 mL; 43.9 mmol). The mixture was allowed to warm to rt overnight and quenched with 25% NH₄OAc. The solvent was removed and the residue stirred in ethyl acetate. The solid was filtered and the filtrate concentrated and purified over silica gel (eluting with 8% MeOH in DCM) to yield the title compound. ¹H NMR (400 MHz, acetone-d₆): δ 6.39 (br, 2H), 1.9 (q, 4H), 0.89 (t, 6H).

### Step 3: 7-Formyl-4-phenylquinoline-2-carbonitrile

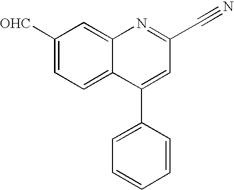

The title compound was prepared from 7-methyl-4-phenylquinoline-2-carbonitrile according to Steps 6 and 7 of Example 3. ¹H NMR (400 MHz, acetone-d₆): δ 10.39 (s, 1H), 8.80 (s, 1H), 8.21 (s, 2H), 8.07 (s, 1H), 7.66 (m, 5H).

### Step 4: 7-({[5-(1-Ethyl-1-hydroxypropyl)-1,3,4-thiadiazol-2-yl]amino}methyl)-4-phenylquinoline-2-carbonitrile

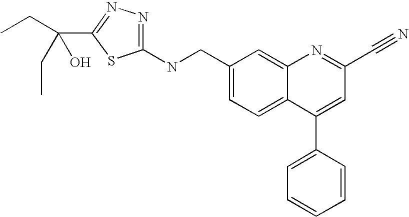

7-Formyl-4-phenylquinoline-2-carbonitrile (0.103 g, 0.4 mmol), 3-(5-amino-1,3,4-thiadiazol-2-yl)pentan-3-ol (0.075 g, 0.4 mmol) and HOAc (0.023 mL) were heated at reflux overnight in toluene (4 mL). The reaction mixture was cooled to rt and diluted with THF (4 mL). Sodium triacetoxyborohydride (0.424 g; 2.0 mmol) was added and heated to 50° C. for 1 h. The mixture was quenched with 25% NH₄OAc and extracted three times with ethyl acetate. The organic phases were combined, dried and purified on silica gel (eluting with 3-5% MeOH in DCM) to yield the title compound. ¹H NMR (400 MHz, acetone-d₆): δ 8.22 (s, 1H), 8.02 (d, 1H), 7.85 (m, 2H), 7.68 (m, 5H), 7.47 (bs, 1H), 4.90 (s, 2H), 4.50 (s, 1H), 1.91 (m, 2H), 1.81 (m, 2H), 0.88 (t, 6H).

Mass spectrum: 430 (M+1).

Following the procedures outlined for Examples 1-6, the following additional examples listed in Table 1 were also prepared.

TABLE 1

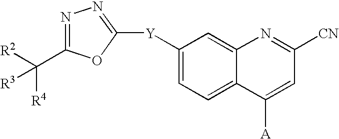

| | | | | | | Mass |
|---|---|---|---|---|---|---|
| | | | | | | spectrum |
| Ex. | R² | R³ | R⁴ | Y | A | (M + 1) |
| 7 | OH | 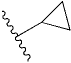 | 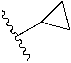 | NHCH₂ | 4-F-Ph | 456 |
| 8 | OH | Et | Et | NHCH₂ | 4-F-Ph | 432 |
| 9 | OH | Et | Et | S | Ph | 417 |
| 10 | OH | Et | Et | N(Me)CH₂ | 3-F-Ph | 446 |
| 11 | OH | Et | Et | NHCH₂ | 3-Me-Ph | 428 |
| 12 | OH | Et | Et | NHCH₂ | 3-F-Ph | 432 |
| 13 | OH | Et | Et | N(Ac)CH₂ | Ph | 456 |
| 14 | OAc | Et | Et | N(Ac)CH₂ | Ph | 498 |
| 15 | OH | Et | Et | NHCH₂ | Ph | 414 |

1. A compound of structural Formula I: 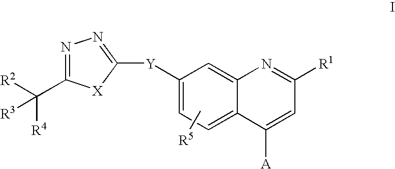
and pharmaceutically acceptable salts and solvates thereof, wherein:
n is 0, 1, or 2;
“A” is selected from the group consisting of
(a) a 5-membered aromatic ring containing (i) one or more carbon atoms, (ii) one heteroatom selected from oxygen and sulfur, and (iii) zero, one, two or three nitrogen atoms,
(b) a 5-membered aromatic ring containing one or more carbon atoms and from one to four nitrogen atoms,
(c) a 6-membered aromatic ring containing carbon atoms and one, two or three nitrogen atoms;
(d) a bicyclic aromatic ring system selected from benzothienyl, indolyl, quinolinyl and naphthalenyl;
(e) phenyl, and
(f) benzyl;
wherein A is optionally mono- or di-substituted with a substituent independently selected at each occurrence from the group consisting of (i) fluorine, (ii) chlorine, (iii) C₁₋₃ alkyl optionally substituted with one to five fluorines, (iv) C₁₋₃ alkoxy optionally substituted with one to five fluorines, (v) C₃₋₆ cycloalkyloxy, (vi) —CH₂OH, (vii) —COOR¹¹, (viii) cyano, (ix) hydroxy, and (x) —NR⁹R¹⁰;
Y is selected from:
(a) NR⁶—CHR⁷ wherein the nitrogen in Y is linked to the 5-membered heterocyclic moiety of Formula I and the carbon in Y is linked to the quinoline moiety of Formula I; and (b) S(O)ₙ;
X is selected from O and S;
each R¹¹ is independently selected from the group consisting of hydrogen, C₁₋₆ alkyl, and C₃₋₆ cycloalkyl;
R¹ is selected from the group consisting of cyano and —CONR¹¹R¹¹;
R² is selected from the group consisting of hydrogen, hydroxy, fluorine, C₁₋₃ alkyl, C₁₋₃ alkoxy, and C₁₋₃ alkylcarbonyloxy;
R³ is selected from the group consisting of hydrogen, C₁₋₆ alkyl optionally substituted with R⁸ or one to five fluorines, C₂₋₆ alkenyl, C₃₋₆ cycloalkyl, C₅₋₇ cycloalkenyl, and -Z;
R⁴ is selected from the group consisting of hydrogen, C₁₋₆ alkyl optionally substituted with R⁸ or one to five fluorines, C₂₋₆ alkenyl, C₃₋₆ cycloalkyl, C₅₋₇ cycloalkenyl, and -Z;
or R³ and R⁴ together represent oxo;
or R³ and R⁴ are joined together with the carbon to which they are attached to form a ring selected from C₃₋₆ cycloalkyl and C₅₋₇ cycloalkenyl, provided that when R³ and R⁴ are joined together with the carbon to which they are attached to form a C₅₋₇ cycloalkenyl ring, there is no double bond at the C-1 position in the ring;
or R², R³, and R⁴ are joined together with the carbon to which they are attached to form a cycloalkenyl ring selected from: 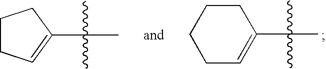
R⁵ is selected from the group consisting of hydrogen, C₁₋₆ alkyl, C₃₋₆ cycloalkyl, and halogen;
R⁶ is selected from the group consisting of hydrogen, C₁₋₄ alkyl, C₁₋₄ alkylcarbonyl, and benzoyl optionally substituted with C₁₋₄ alkyl;
R⁷ is selected from the group consisting of (a) hydrogen, (b) C₁₋₄ alkyl, (c) C₃₋₆ cycloalkyl, (d) phenyl optionally mono- or di-substituted with a substituent independently selected at each occurrence from the group consisting of C₁₋₄ alkyl and fluorine, and (e) a 5-membered aromatic ring containing (i) one or more carbon atoms, (ii) one heteroatom selected from oxygen and sulfur, and (iii) zero, one, two or three nitrogen atoms;
R⁸ is selected from the group consisting of —COOR¹¹, —C(O)H, cyano, —CR¹¹R¹¹OH, —OR¹¹, C₁₋₆ alkylthio, and C₃₋₆ cycloalkylthio;
R⁹ is selected from the group consisting of hydrogen, C₁₋₆ alkyl, C₃₋₆ cycloalkyl, and —COOR^{a};
R^{a} is C₁₋₆ alkyl or C₃₋₆ cycloalkyl;
R¹⁰ is selected from the group consisting of hydrogen, C₁₋₆ alkyl, and C₃₋₆ cycloalkyl; and
Z is selected from the group consisting of
(a) a 5-membered aromatic ring containing (i) one or more carbon atoms, (ii) one heteroatom selected from oxygen and sulfur, and (iii) zero, one, two or three nitrogen atoms,
(b) a 5-membered aromatic ring containing one or more carbon atoms and from one to four nitrogen atoms,
(c) a 6-membered aromatic ring containing carbon atoms and one, two or three nitrogen atoms;
(d) phenyl,
(e) benzyl, and (f) —CH₂-dioxolanyl;
and wherein Z is optionally mono- or di-substituted with a substituent independently selected at each occurrence from the group consisting of (i) fluorine, (ii) chlorine, (iii) C₁₋₃ alkyl optionally substituted with one to five fluorines, (iv) C₁₋₃ alkoxy optionally substituted with one to five fluorines, (v)
C₃₋₆ cycloalkyloxy, (vi) —CH₂OH, (vii) —COOR¹¹, (viii) cyano, and (ix) —NR⁹R¹⁰.

2. The compound of claim 1 of structural Formula II: 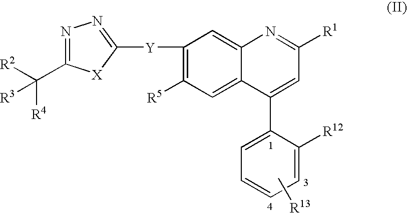
wherein R¹² is selected from the group consisting of hydrogen and fluorine; and
R¹³ is a substituent at the 3- or 4-position of the phenyl ring and is selected from the group consisting of (i) fluorine, (ii) C₁₋₃ alkyl optionally substituted with one to five fluorines, (iii) C₁₋₃ alkoxy optionally substituted with one to five fluorines, (iv) C₃₋₆ cycloalkyloxy, (v) —CH₂OH, (vi) —COOR¹¹, (vii) cyano, and (viii) —NR⁹R¹⁰.

3. The compound of claim 1 of structural Formula II: 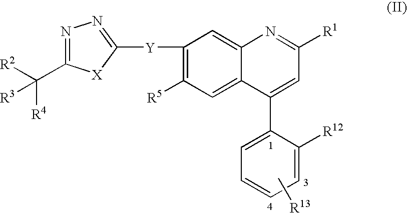
wherein R¹² is selected from the group consisting of hydrogen and fluorine; and
R¹³ is selected from the group consisting of hydrogen, fluorine, trifluoromethoxy, difluoromethoxy, cyano, methyl, and methoxy.

4. The compound of claim 3 wherein R¹³ is hydrogen or fluorine.

5. The compound of claim 2 of structural Formula III:

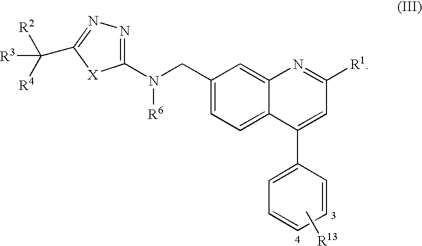

6. The compound of claim 2 of structural Formula IV:

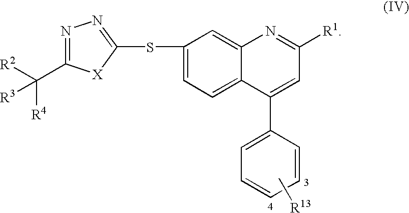

7. The compound of claim 1 wherein A is selected from the group consisting of:
a) a 5-membered aromatic ring containing (i) one or more carbon atoms, (ii) one heteroatom selected from oxygen and sulfur, and (iii) zero, one, two or three nitrogen atoms,
b) a 5-membered aromatic ring containing one or more carbon atoms and from one to four nitrogen atoms,
c) a 6-membered aromatic ring containing carbon atoms and one, two or three nitrogen atoms, and
d) phenyl,
and wherein A is unsubstituted, mono- or di-substituted with a substituent independently selected at each occurrence from the group consisting of (i) fluorine, (ii) chlorine, (iii) C₁₋₃ alkyl optionally substituted with one to five fluorines, (iv) C₁₋₃ alkoxy optionally substituted with one to five fluorines, (v) C₃₋₆ cycloalkyloxy, (vi) —CH₂OH, (vii) —COOR¹¹, (viii) cyano, (ix) hydroxy, and (x) —NR⁹R¹⁰.

8. The compound of claim 7 wherein A is phenyl, optionally substituted at the 3- or 4-position with a substituent independently selected from fluorine, trifluoromethoxy, difluoromethoxy, cyano, methyl, and methoxy and optionally substituted at the 2-position with fluorine.

9. The compound of claim 8 wherein A is 4-fluorophenyl.

10. The compound of claim 1 selected from the group consisting of:
4-(4-fluorophenyl)-7-[({5-[1-hydroxy-1-(trifluoromethyl)propyl]-1,3,4-oxadiazol-2-yl}amino)methyl]quinoline-2-carbonitrile;
7-[({5-[dicyclopropyl(hydroxy)methyl]-1,3,4-oxadiazol-2-yl}amino)methyl]-4-(3-fluorophenyl)quinoline-2-carbonitrile;
7-[({5-[dicyclopropyl(hydroxy)methyl]-1,3,4-oxadiazol-2-yl}amino)methyl]-4-(4-fluorophenyl)quinoline-2-carbonitrile;
7-({[5-(1-ethyl-1-hydroxypropyl)-1,3,4-oxadiazol-2-yl]amino}methyl)-4-(4-fluorophenyl)quinoline-2-carbonitrile;
4-phenyl-7-{[(5-propionyl-1,3,4-oxadiazol-2-yl)amino]methyl}quinoline-2-carbonitrile;
7-{[5-(1-ethyl-1-hydroxypropyl)-1,3,4-oxadiazol-2-yl]thio}-4-(3-methylphenyl)quinoline-2-carbonitrile;
7-{[5-(1-ethyl-1-hydroxypropyl)-1,3,4-oxadiazol-2-yl]thio}-4-phenylquinoline-2-carbonitrile;
7-{[[5-(1-ethyl-1-hydroxypropyl)-1,3,4-oxadiazol-2-yl](methyl)amino]methyl}-4-(3-fluorophenyl)quinoline-2-carbonitrile;
7-({[5-(1-ethyl-1-hydroxypropyl)-1,3,4-oxadiazol-2-yl]amino}methyl)-4-(3-methylphenyl)quinoline-2-carbonitrile;
7-({[5-(1-ethyl-1-hydroxypropyl)-1,3,4-oxadiazol-2-yl]amino}methyl)-4-(3-fluorophenyl)quinoline-2-carbonitrile;
N-{[2-cyano-4-(3-fluorophenyl)quinolin-7-yl]methyl}-N-[5-(1-ethyl-1-hydroxypropyl)-1,3,4-oxadiazol-2-yl]acetamide;
N-[(2-cyano-4-phenylquinolin-7-yl)methyl]-N-[5-(1-ethyl-1-hydroxypropyl)-1,3,4-oxadiazol-2-yl]acetamide;
1-(5-{acetyl[(2-cyano-4-phenylquinolin-7-yl)methyl]amino}-1,3,4-oxadiazol-2-yl)-1-ethylpropyl acetate;
7-({[5-(1-ethyl-1-hydroxypropyl)-1,3,4-oxadiazol-2-yl]amino}methyl)-4-phenylquinoline-2-carbonitrile; and
7-({[5-(1-ethyl-1-hydroxypropyl)-1,3,4-thiadiazol-2-yl]amino}methyl)-4-phenylquinoline-2-carbonitrile;
and pharmaceutically acceptable salts and solvates thereof.

11. A method of preventing the synthesis, the action, or the release of leukotrienes in a mammal which comprises administering to said mammal an effective amount of a compound of claim 1.

12. The method of claim 11 wherein said mammal is a human.

13. A method of treating an inflammatory condition in a patient which comprises administering to a mammal in need of such treatment a therapeutically effective amount of a compound of claim 1.

14. A method of treating atherosclerosis comprising administering a therapeutically effective amount of a compound of claim 1 to a patient in need of such treatment.

15. A method for preventing or reducing the risk of an atherosclerotic disease event comprising administering a prophylactically effective amount of a compound of claim 1 to a patient at risk for having an atherosclerotic disease event.

16. A method for the prophylaxis or treatment of asthma comprising administering a therapeutically effective amount of a compound of claim 1 to a patient in need of such treatment.

17. A method for treating allergic rhinitis comprising administering a therapeutically effective amount of a compound of claim 1 to a patient in need of such treatment.

18. A method for treating COPD comprising administering a therapeutically effective amount of a compound of claim 1 to a patient in need of such treatment.

19. A pharmaceutical composition comprised of a therapeutically effective amount of a compound of claim 1 and a pharmaceutically acceptable carrier.

20. A pharmaceutical composition comprised of a therapeutically effective amount of a compound of claim 1, at least one additional therapeutically active agent and a pharmaceutically acceptable carrier.

21-32. (canceled)
